(19) United States
(12) Patent Application Publication
(10) Pub. No.: US 2013/0316349 A1
(43) Pub. Date: Nov. 28, 2013

(54) N-METHYLPURINE DNA GLYCOSYLASE AND POLYMERASE BETA AS BIOMARKERS FOR ALKYLATOR CHEMOTHERAPY POTENTIATION

(71) Applicants: Robert W. Sobol, JR., Pittsburgh, PA (US); Jiangbo Tang, Drexel Hill, PA (US)

(72) Inventors: Robert W. Sobol, JR., Pittsburgh, PA (US); Jiangbo Tang, Drexel Hill, PA (US)

(73) Assignee: University of Pittsburgh - Of the Commonwealth System of Higher Education, Pittsburgh, PA (US)

(21) Appl. No.: 13/961,526

(22) Filed: Aug. 7, 2013

Related U.S. Application Data
(63) Continuation of application No. 13/077,502, filed on Mar. 31, 2011.
(60) Provisional application No. 61/320,572, filed on Apr. 2, 2010.

(51) Int. Cl. C12Q 1/68; G01N 33/68

(52) U.S. Cl. CPC C12Q 1/6881; G01N 33/68
USPC 435/6.11; 435/7.4

(57) ABSTRACT

Described herein is the finding that polymerase β (Polβ) and N-methylpurine DNA glycosylase (MPG) can be used as biomarkers to evaluate the sensitivity of a subject to combination therapy that includes treatment with either temozolomide (TMZ) and methoxyamine, or TMZ and a poly(ADP-ribose) polymerase (PARP) inhibitor. Thus, provided herein is a method of determining if a subject will be sensitive to TMZ and methoxyamine, or TMZ and a PARP inhibitor by measuring expression of Polβ and MPG in a sample from the subject and comparing expression of Polβ and MPG in the sample to a control. A decrease in expression of Polβ and an increase in expression of MPG relative to the control indicates the subject is sensitive to TMZ and methoxyamine, or sensitive to TMZ and the PARP inhibitor.

### CROSS REFERENCE TO RELATED APPLICATIONS

This is a continuation of U.S. application Ser. No. 13/077,502, filed Mar. 31, 2011, which claims the benefit of U.S. Provisional Application No. 61/320,572, filed Apr. 2, 2010. The above-listed applications are herein incorporated by reference in their entirety.

### ACKNOWLEDGMENT OF GOVERNMENT SUPPORT

This invention was made with government support under grant numbers CA132385 and CA148629, awarded by the National Institutes of Health. The government has certain rights in the invention.

### FIELD

This disclosure concerns biomarkers that can be used to determine a subject's sensitivity to chemotherapeutic agents, particularly alkylating agents used in combination with modulators of the base excision repair pathway.

### BACKGROUND

Temozolomide (TMZ) is an oral chemotherapeutic agent approved for the treatment of anaplastic astrocytoma and newly diagnosed glioblastoma (Mrugala and Chamberlain, Nat. Clin. Pract. Oncol., 5:476-489, 2008). TMZ has also shown clinical activity in metastatic melanoma and is under clinical evaluation for its use in other cancers, including leukemia, lymphoma, aerodigestive tract, pancreatic and neuroendocrine tumors, as well as cancers that have metastasized to the brain (Tentori and Graziani, Curr. Med. Chem., 16:245-257, 2009). TMZ causes cancer cell cytotoxicity by methylating genomic DNA, producing cytotoxic and/or mutagenic abnormal DNA bases (Sobol, Schwab M, ed. Encyclopedia of Cancer, Berlin, Heidelberg, New York: Springer, 2008). The major site of methylation is the N7 position of guanine (>70%) followed by the N3 position of adenine (9.2%) and the O⁶ atom of guanine (5%) (Sobol, Schwab M, ed. Encyclopedia of Cancer, Berlin, Heidelberg, New York: Springer, 2008). However, the ability of cancer cells to recognize and repair those DNA lesions confers chemotherapeutic resistance and imposes a negative impact on therapeutic efficacy (Sarkaria et al., Clin. Cancer Res., 14:2900-2908, 2008).

The majority of TMZ-induced DNA lesions, including the N7-methyl guanine and N3-methyl adenine, are repaired by the base excision repair (BER) pathway (Sobol, Schwab M, ed. Encyclopedia of Cancer, Berlin, Heidelberg, New York: Springer, 2008), while the O⁶-methyl adduct of guanine is directly removed by O-6-methylguanine-DNA methyltransferase (MGMT). Although O⁶-methylguanine constitutes only a small proportion of the base lesions produced by TMZ, it is the most cytotoxic lesion and constitutes a significant part of TMZ-induced cytotoxicity (Tentori and Graziani, Curr. Med. Chem., 16:245-257, 2009). Since O⁶-methylguanine-induced cytotoxicity is mediated through the mismatch repair (MMR) pathway, sensitivity to TMZ requires both low MGMT activity and functional MMR (Tentori and Graziani, Curr. Med. Chem., 16:245-257, 2009).

Although a proportion of gliomas have been found to be lacking the expression of MGMT due to hyper-methylation of MGMT promoters, and thus possessing diminished DNA repair activity, at least half of glioblastoma multiforme (GBM) express MGMT and the expression is associated with resistance to chemotherapy and poor prognosis (Hegi et al., N. Eng. J. Med., 352:997-1003, 2005). Loss of the mismatch repair protein MSH6 due to tumor-specific mutations has also been shown to be associated with glioblastoma recurrences post-radiation and TMZ treatment (Cahill et al., Clin. Cancer Res., 13:2038-2045, 2007). Therefore, it is important to either overcome resistance rendered by the activity of MGMT or find an alternative that improves efficacy of TMZ in the presence of MGMT activity. Pharmacological inhibition of the BER pathway, which repairs the N7-methylguanine and N3-methyladenine lesions produced by TMZ (among other DNA lesions), has been shown to enhance TMZ-induced cytotoxicity independent of MGMT status (Adhikari et al., Anticancer Agents Med Chem., 8:351-357, 2008).

The repair of TMZ-induced base damage by the BER pathway starts with the recognition and removal of the damaged bases by a specific DNA glycosylase N-methylpurine DNA glycosylase (MPG), also known as alkyladenine DNA glycosylase (Wood et al., Science, 291:1284-1289, 2001). The abasic site (AP site) produced following the action of MPG is then hydrolyzed by AP endonuclease (APE1) leading to incision of the damaged DNA strand, leaving a 3′OH group and 5′ deoxy-ribose phosphate (5′ dRP) group (Almeida and Sobol, DNA Repair (Amst) 6:695-711, 2007). Poly(ADP-ribose) polymerase 1 (PARP1), together with PARP2 and Poly(ADP-ribose) glycohydrolase (PARG), recognizes the DNA strand interruption and facilitates the recruitment of subsequent BER proteins, including the BER scaffold protein XRCC1 and DNA polymerase β (Pol β) (Almeida and Sobol, DNA Repair (Amst) 6:695-711, 2007). Pol β subsequently hydrolyzes the 5′ dRP moiety and inserts a single nucleotide, preparing the strand for ligation by a complex of DNA Ligase Ma and XRCC1 to complete the repair process (Sobol et al., Nature, 405:807-810, 2000).

Enhanced sensitivity to alkylating agents has been observed by modulating the BER pathway in preclinical studies, suggesting BER modulation is an attractive target for chemotherapy potentiation (Kinsella, Clin. Cancer Res., 15:1853-1859, 2009). Currently, several BER proteins are under active investigation as potential targets for chemotherapy sensitization, including APE1 (Liu and Gerson, Curr. Opin. Investig. Drugs 5:623-627, 2004), PARP1 (Kinsella, Clin. Cancer Res. 15:1853-1859, 2009), PARG (Tentori et al., Eur. J. Cancer 41:2948-2957, 2005), and Pol β (Trivedi et al., Cancer Res. 65:6395-6400, 2005; Trivedi et al., Mol Pharmacol 2008; Mizushina et al., Molecules 14:102-121, 2009). Methoxyamine (MX) is a small molecule that specifically inhibits BER (Rosa et al., Nucleic Acids Res. 19:5569-5574, 1991). It is currently under a phase I clinical trial under the name of TRC102. Methoxyamine inhibits repair of AP sites by binding and modifying the DNA substrate, AP sites, rather than directly inhibiting the enzyme APE1. AP sites modified by MX are refractory to APE1, preventing its processing by ensuing steps of BER and is cytotoxic (Yan et al., Clin. Cancer Res. 13:1532-1539, 2007). Methoxyamine potentiates a wide range of DNA damaging agents that produce AP sites regardless of the status of mismatch repair (MMR), MGMT, and p53 (Liu and Gerson, Eur. J. Cancer 41:2948-2957, 2005).

PARP1 is the founding member of a large family of poly(ADP-ribose) polymerases with 17 members identified (Ame et al., Bioessays 26:882-893, 2004). It is the primary enzyme catalyzing the transfer of ADP-ribose units from NAD to target proteins including PARP1 itself. Under normal physiologic conditions, PARP1 facilitates the repair of DNA base lesions by helping recruit BER proteins XRCC1 and Polβ (Dantzer et al., Methods Enzymol. 409:493-510, 2006). Inhibition of PARP1 results in decreased repair of DNA base damage and increased sensitivity of cells to alkylating agents, which makes it an attractive and effective target for chemotherapy sensitization (Fisher et al., Mol. Cell Biol. 27:5597-5605, 2007). Many PARP inhibitors have been developed and tested in several tumor types (Ratnam and Low, Clin. Cancer Res. 13:1383-1388, 2007). They have been shown to enhance the antitumor activity of TMZ against glioma (Tentori et al., Glia 40:44-54, 2002; Tentori et al., Clin. Cancer Res. 9:5370-5379, 2003; Cheng et al., Mol. Cancer Ther. 4:1364-1368, 2005), leukemia (Tentori et al., Cancer Chemother. Pharmacol. 47:361-369, 2001), lung (Miknyoczki et al., Mol. Cancer Ther. 2:371-382, 2003; Calabrese et al., J. Natl. Cancer Inst. 96:56-67, 2004) and colon (Calabrese et al., J. Natl. Cancer Inst. 96:56-67, 2004; Calabrese et al., Clin. Cancer Res. 9:2711-2718, 2003; Curtin et al., Clin. Cancer Res. 10:881-889, 2004) carcinoma.

PARG is the main enzyme responsible for degradation of poly ADP-ribose (PAR) in vivo via endo- and exo-glycosidic cleavage. Although complete ablation of PARG activity leads to early embryonic lethality, embryonic stem cells derived from PARG null mouse (Koh et al., Proc. Natl. Acad. Sci. USA 101:17699-17704, 2004) and cells from PARG₁₁₀ (one of three isoforms of PARG) deficient mice (Cortes et al., Mol. Cell Biol. 24:7163-7178, 2004) have been shown to be sensitive to alkylating agents and ionizing radiation. It has also been shown that inhibition of PARG activity sensitized malignant melanoma to TMZ in a mouse model (Tentori et al., Eur. J. Cancer 41:2948-2957, 2005). Over-expression of MPG has been reported to sensitize human breast cancer cells (Trivedi et al., Mol. Pharmacol. 2008; Rinne et al., Mol. Cancer Ther. 3:955-967, 2004), osteosarcoma cells (Wang et al., Zhonghua Bing Li Xue Za Zhi 35:352-356, 2006), and ovarian cancer cells (Fishel et al., Clin. Cancer Res. 13:260-267, 2007) to chemotherapeutic agent TMZ. The increased alkylation sensitivity has been shown to be the result of increased repair initiation of the non-toxic N7-methyl guanine lesion (Rinne et al., Nucleic Acids Res. 33:2859-2867, 2005), saturating the rating-limiting enzyme Polβ resulting in accumulation of cytotoxic 5′ dRP repair intermediates (Trivedi et al., Cancer Res. 65:6394-6400, 2005). The mechanism of 5′ dRP accumulation induced cytotoxicity has recently been elucidated to be a PARP activation dependent, cellular energy metabolism related cell death mechanism (Tang et al., Mol. Cancer Res. 8(1):67-79, 2010).

### SUMMARY

Described herein is the finding that DNA Polymerase β (Polβ) and N-methylpurine DNA glycosylase (MPG) can be used as biomarkers to evaluate the sensitivity of a subject to combination therapy that includes treatment with either temozolomide (TMZ) and methoxyamine, or TMZ and a poly(ADP-ribose) polymerase (PARP) inhibitor.

Provided herein is a method of determining if a subject will be sensitive to TMZ and methoxyamine, or TMZ and a PARP inhibitor. In some embodiments, the method includes measuring expression of Polβ and MPG in a sample from the subject and comparing expression of Polβ and MPG in the sample to a control. A decrease in expression of Polβ and an increase in expression of MPG relative to the control indicates the subject is sensitive to TMZ and methoxyamine, or sensitive to TMZ and the PARP inhibitor.

The foregoing and other objects, features, and advantages of the invention will become more apparent from the following detailed description, which proceeds with reference to the accompanying figures.

### BRIEF DESCRIPTION OF THE FIGURES

FIGS. 1A-1F: Over-expression of MPG in LN428 cells dramatically increases MX-induced potentiation of TMZ. (FIG. 1A) MPG over-expression as determined by immunoblot analysis of nuclear proteins isolated from the LN428 or MPG over-expressing LN428 cells. Expression levels of BER protein Polβ and APE1 are also shown. PCNA expression is shown as a loading control. (FIG. 1B) MPG over-expression as determined by qRT-PCR analysis in LN428 cells and LN428/MPG cells (over expresses MPG). (FIG. 1C) A schematic diagram showing the mechanism of the molecular beacon assay that is used in measuring glycosylase activity of MPG. (FIG. 1D) Increased DNA glycosylase activity in MPG over-expressing LN428/MPG cells as determined by the DNA Glycosylase Molecular Beacon Activity Assay. DNA glycosylase activity specific for removal of the MPG substrate εA was measured in nuclear lysates from the control cell line (LN428) and the MPG over-expression cell line (LN428/MPG). Each lysate was analyzed using either the control-beacon or the εA-beacon: LN428 lysates (control-beacon, filled squares; εA-beacon, open squares) and LN428/MPG lysates (control-beacon, open circles; εA-beacon, filled circles). Results are reported as the mean fluorescence response unit of three independent experiments±the standard error of the mean. (FIG. 1E) MPG over-expression increases MX-induced potentiation of TMZ in LN428 cells. LN428 cells (triangle) or MPG over-expressing LN428 cells (inverted triangle) were cultured in 96-well plates for 24 hours prior to exposure to MX (filled symbols). Following exposure to MX (60 mM) for 30 minutes, cells were treated with TMZ together with MX (30 mM) for 48 hours. Viable cells were determined using a modified MTT assay. Plots show the % viable cells as compared to untreated (control) cells. Means are calculated from quadruplicate values in each experiment. Results indicate the mean±S.E. of three independent experiments. (FIG. 1F) Over-expression of glycosylase dead mutant MPG (N169D) in LN428 cells does not increase MX-induced potentiation of TMZ. 24 hours after seeding into 96-well plates, LN428 cells over-expressing mutant MPG (N169D) were treated with (triangle) or without (open circle) MX (60 mM) for 30 minutes. Following MX pre-treatment, cells were exposed to TMZ in the presence (triangle) or absence (open circle) of MX (30 mM) for additional 48 hours. Viable cells were counted and results were reported as in FIG. 1E.

FIGS. 2A-2E: Over-expression of BER protein Polβ but not APE1 reverses MX-induced potentiation of TMZ in MPG over-expressing LN428/MPG cells. (FIG. 2A) Over-expression of WT Pol β or lyase activity dead mutant (K72A) of Polβ in MPG over-expressing LN428/MPG cells as determined by immunoblot analysis of nuclear protein extracted from LN428/MPG-VC (vector control, lane 1), LN428/MPG/Flag-Pol β-WT (clone 1 and 6 stably over-express Flag tagged WT Pol β, lane 4 & 5), and LN428/MPG/Flag-Pol β-K72A (clone 5 and 16 stably over-express Flag tagged mutant Polβ, lanes 2 and 3) cells. PCNA is shown as a loading control. (FIG. 2B) Over-expression of Polβ reverses MX-induced potentiation of TMZ in MPG overexpressing LN428/MPG cells. Cell viability assays were performed and results were reported as in FIG. 1E. Shown are LN428/MPG/Flag-Polβ-WT clone 1 (triangle), LN428/MPG/FlagPolβ-WT clone 6 (inverted triangle), cells treated with TMZ only (open symbols), cells treated with TMZ and MX (filled symbols). The dotted line with diamond symbols shows LN428/MPG cells treated with MX and TMZ, as shown in FIG. 1E. (FIG. 2C) Over-expression of mutant Pol β (K72A) does not reverse MX-induced potentiation of TMZ in MPG over-expressing LN428/MPG cells. Cell viability assays were performed and results were reported as in FIG. 1E. Shown are LN428/MPG/Flag-Pol β-K72A c5 (triangle), LN428/MPG/Flag-Pol β-K72A c16 (inverted triangle), cells treated with TMZ only (open symbols), cells treated with TMZ and MX (filled symbols). The dotted line with diamond symbols shows LN428/MPG cells treated with MX and TMZ as shown in FIG. 1E. (FIG. 2D) Immunoblot shows over-expression of Flag-tagged APE1 in the LN428/MPG cells. Lane 1: LN428/MPG/vector control; lane 2: LN428/MPG/Flag-APE1 clone 4; lane 3: LN428/MPG/Flag-APE1 clone 8. PCNA was used as a loading control. (FIG. 2E) Over-expression of APE1 does not reverse MX-induced potentiation of TMZ in MPG over-expressing LN428/MPG cells. Cell viability assays were performed and results were reported as in FIG. 1E. Shown are LN428/MPG/Flag-APE1 clone 4 (triangle), LN428/MPG/Flag-APE1 clone 8 (inverted triangle), cells treated with TMZ only (open symbols), cells treated with TMZ and MX (filled symbols).

FIGS. 3A-3G: Over-expression of MPG increases PARG KD-induced potentiation of TMZ in LN428/MGMT cells. (FIG. 3A) MGMT expression as determined by immunoblot analysis of nuclear protein isolated from LN428 cells (lane 1) and T98G cells (lane 2; used as a positive control). PCNA expression is shown as a loading control. (FIG. 3B) MGMT over-expression as determined by immunoblot analysis of nuclear protein isolated from LN428 cells over-expressing MGMT (lane 1) and T98G cells (lane 2; used as a positive control). PCNA expression is shown as a loading control. (FIG. 3C) Over-expression of MGMT provided the sensitive LN428 cells resistance to TMZ. Cell viability assays were performed and results were reported as in FIG. 1E. Shown are LN428, empty circle; and LN428/MGMT, empty rectangle. (FIG. 3D) A schematic diagram showing the five PARG shRNA constructs targeting PARG mRNA. (FIG. 3E) Decreased PARG mRNA expression levels induced by the five shRNA constructs targeting PARG. Results are reported as the mean±S.E. of three independent qRT-PCR experiments. (FIG. 3F) PARG KD induces delayed degradation of PAR in LN428/MPG cells following exposure to 1.5 mM TMZ as demonstrated by immunoblot analysis. (FIG. 3G) PARG KD significantly reduced cell survival following exposure to 300 μM TMZ in cells over expressing MPG (LN428/MGMT/MPG) as determined by long-term cell survival assay, and sensitization was not observed in LN428/MGMT cells with low MPG expression level.

FIGS. 4A-4E: Over-expression of MPG increases PARP inhibitor PJ34-induced potentiation of TMZ in glioma cells with over expressed MGMT. (FIG. 4A) Over-expression of MPG in LN428/MGMT cells substantially increased PJ34-induced potentiation of TMZ as measured by long-term cell survival assays. Shown are LN428/MGMT cells (triangle); LN428/MGMT/MPG cells (reversed triangle); TMZ treatment only (empty symbols); and PJ34 and TMZ treatment (filled symbols). Results were calculated as percentage survival relative to non TMZ treated control cells (% control) and reported as the mean±S.E. of three independent experiments. (FIG. 4B) Over-expression of MPG in T98G cells as shown by Western blot (T98G, lane 1; T98G/MPG, lane 2). Tubulin was used as a loading control. (FIG. 4C) Over-expression of MPG in T98G cells as shown by qRT-PCR. (FIG. 4D) Over-expression of MPG in T98G cells significantly increased A8T-888-induced potentiation of TMZ as measured by long-term cell survival assays (white bars, no TMZ treatment controls; lined bars, 50 μM TMZ treatment; black bars, 100 μM TMZ treatment). Results were calculated and reported as in FIG. 4A. Statistics, student-t test, *: p<0.05; **: p<0.01. (FIG. 4E) Polβ depletion by shRNA combined with over-expression of MPG in T98G cells significantly increased ABT-888-induced potentiation of TMZ. No TMZ treatment controls (white bars); 25 μM TMZ treatment (grey bars); 50 μM TMZ treatment (lined bars). Results were calculated and reported as in FIG. 4A. Statistics comparing between treatments with or without ABT-888, student-t test, **: p<0.01.

FIG. 5: MPG over-expression increases MX-induced potentiation of TMZ in LN428 cells. LN428 cells (white triangle) or LN428/MPG cells (inverted white triangle) were cultured in 96-well plates for 24 hours prior to exposure to MX (filled symbols). Following exposure to MX (60 mM) for 30 minutes, cells were treated with TMZ together with MX (30 mM) for 48 hours. Viable cells were determined using a modified MTT assay. Plots show the % viable cells as compared to untreated (control) cells. Means are calculated from quadruplicate values in each experiment. Results indicate the mean±S.E. of three independent experiments.

FIGS. 6A-6D: (FIG. 6A) MGMT promoter methylation analysis of Bisulfite-treated DNA used for PCR in the DNA extracted from LN428 cells (Group 2, U: unmethylated and M: methylated), T98G cells (Group 3, U: unmethylated and M: methylated), Universal unmethylated DNA as negative control DNA (Group 1, U: unmethylated) and Universal methylated DNA as a positive control (Group 1, M: methylated). (FIG. 6B) Over-expression of APE1 in LN428/MPG cells as shown by qRT-PCR. Two clones (clone #4 and clone #8) are shown. (C and D) qRT-PCR results showing PARG KD in LN428/MGMT cells (FIG. 6C) and LN428/MPG/MGMT cells (FIG. 6D). Results are reported as the mean±S.E. of three independent experiments.

FIGS. 7A-7D: Expression profile of MPG, PARP1 and Polβ in established glioma cell lines. (FIGS. 7A-7C) The relative expression of mRNA for (FIG. 7A) MPG, (FIG. 7B) Polβ and (FIG. 7C) PARP1 in GBM cell lines, as indicated in the figure, was measured by quantitative RT-PCR using an Applied Biosystems StepOnePlus™ system, normalizing to the LN428 cell line across samples. Analysis of mRNA expression was conducted as per the manufacturer (ΔΔC_{T} method), normalized within each sample to the expression of human β-actin (part #4333762T). (FIG. 7D) MPG, Polβ and PARP1 expression as determined by immunoblot analysis of nuclear protein extracted from the indicated cells. PCNA expression is shown as a loading control.

FIGS. 8A-8C: Relative mRNA expression levels of MPG, PARP1 and Polβ in GBM tumors as compared to normal brain. (FIGS. 8A-8C) The relative expression of mRNA for (FIG. 8A) MPG, (FIG. 8B) Polβ and (FIG. 8C) PARP1 in GBM tumors (tissue #4-16) as indicated in the figure, was measured by quantitative RT-PCR using an Applied Biosystems StepOnePlus™ system, normalizing to the normal brain sample (tissue #20) across tissue samples. Analysis of mRNA expression was conducted as per the manufacturer (ΔΔC_{T} method), normalized within each sample to the expression of human β-actin (part #4333762T).

### Sequence Listing

The nucleic acid sequences listed in the accompanying sequence listing are shown using standard letter abbreviations for nucleotide bases as defined in 37 C.F.R. 1.822. Only one strand of each nucleic acid sequence is shown, but the complementary strand is understood as included by any reference to the displayed strand. The Sequence Listing is submitted as an ASCII text file, created on Jul. 28, 2013, 2.93 KB, which is incorporated by reference herein. In the accompanying sequence listing:

SEQ ID NOs: 1 and 2 are the nucleotide sequences of oligonucleotides used in the molecular beacon MPG activity assay.

SEQ ID NOs: 3 and 4 are the nucleotide sequences of primers specific for methylated human MGMT promoters.

SEQ ID NOs: 5 and 6 are the nucleotide sequences of primers specific for unmethylated human MGMT promoters.

SEQ ID NOs: 7 and 8 are the nucleotide sequences of primers for amplification of human MGMT cDNA.

SEQ ID NOs: 9-13 are the nucleotide sequences of PARG-specific shRNAs.

### DETAILED DESCRIPTION

### I. Abbreviations

AU Arbitrary unit

BER Base excision repair

cDNA Complementary DNA

DNA Deoxyribonucleic acid

DSB Double strand break

FACS Fluorescence activated cell sorting

FBS Fetal bovine serum

GBM Glioblastoma multiforme

HIV Human immunodeficiency virus

HPV Human papillomavirus

KD Knockdown (or depletion)

MGMT O-6-methylguanine-DNA methyltransferase

miRNA MicroRNA

MMR Mismatch repair

MPG N-methylpurine DNA glycosylase

mRNA Messenger RNA

MSP Methylation-specific PCR

MX Methoxyamine

ORF Open reading frame

PCR Polymerase chain reaction

PAR Poly(ADP-ribose)

PARG Poly(ADP-ribose) glycohydrolase

PARP Poly(ADP-ribose) polymerase

qRT-PCR Quantitative reverse-transcriptase polymerase chain reaction

RNA Ribonucleic acid

RNAi RNA interference

shRNA Short hairpin RNA

siRNA Small interfering RNA

TMZ Temozolomide

### II. Terms and Methods

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of the disclosure, the following explanations of specific terms are provided:

Administration: The introduction of a composition into a subject by a chosen route. For example, if the chosen route is intravenous, the composition is administered by introducing the composition into a vein of the subject.

Antibody: A protein (or protein complex) that includes one or more polypeptides substantially encoded by immunoglobulin genes or fragments of immunoglobulin genes. The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The basic immunoglobulin (antibody) structural unit is generally a tetramer. Each tetramer is composed of two identical pairs of polypeptide chains, each pair having one light (about 25 kD) and one heavy chain (about 50-70 kD). The N-terminus of each chain defines a variable region of about 100 to 110 or more amino acids primarily responsible for antigen recognition. The terms variable light chain (V_{L}) and variable heavy chain (V_{H}) refer, respectively, to these light and heavy chains.

As used herein, the term antibody includes intact immunoglobulins as well as a number of well-characterized fragments produced by digestion with various peptidases, or genetically engineered artificial antibodies. Thus, for example, pepsin digests an antibody below the disulfide linkages in the hinge region to produce F(ab)′₂, a dimer of Fab which itself is a light chain joined to V_{H}-C_{H} 1 by a disulfide bond. The F(ab)′₂ may be reduced under mild conditions to break the disulfide linkage in the hinge region thereby converting the F(ab)′₂ dimer into an Fab′ monomer. The Fab′ monomer is essentially a Fab with part of the hinge region (see, Fundamental Immunology, W. E. Paul, ed., Raven Press, N.Y., 1993). While various antibody fragments are defined in terms of the digestion of an intact antibody, it will be appreciated that Fab′ fragments may be synthesized de novo either chemically or by utilizing recombinant DNA methodology. Thus, the term antibody as used herein also includes antibody fragments either produced by the modification of whole antibodies or synthesized de novo using recombinant DNA methodologies.

Antibodies for use in the methods of this disclosure can be monoclonal or polyclonal. Merely by way of example, monoclonal antibodies can be prepared from murine hybridomas according to the classical method of Kohler and Milstein (Nature 256:495-497, 1975) or derivative methods thereof. Detailed procedures for monoclonal antibody production are described in Harlow and Lane (Antibodies, A Laboratory Manual, CSHL, New York, 1988). Rabbit monoclonal antibodies can also be generated accordingly to known procedures.

The terms bind specifically and specific binding refer to the ability of a specific binding agent (such as, an antibody) to bind to a target molecular species in preference to binding to other molecular species with which the specific binding agent and target molecular species are admixed. A specific binding agent is said specifically to recognize a target molecular species when it can bind specifically to that target. In one embodiment, the antibody specifically binds PARP, MPG or Polβ, for example.

A single-chain antibody (scFv) is a genetically engineered molecule containing the V_{H} and V_{L} domains of one or more antibody(ies) linked by a suitable polypeptide linker as a genetically fused single chain molecule (see, for example, Bird et al., Science, 242:423-426, 1988; Huston et al., Proc. Natl. Acad. Sci., 85:5879-5883, 1988). Diabodies are bivalent, bispecific antibodies in which V_{H} and V_{L} domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444-6448, 1993; Poljak et al., Structure, 2:1121-1123, 1994). One or more CDRs may be incorporated into a molecule either covalently or noncovalently to make the resultant molecule an immunoadhesin. An immunoadhesin may incorporate the CDR(s) as part of a larger polypeptide chain, may covalently link the CDR(s) to another polypeptide chain, or may incorporate the CDR(s) noncovalently. The CDRs permit the immunoadhesin to specifically bind to a particular antigen of interest. A chimeric antibody is an antibody that contains one or more regions from one antibody and one or more regions from one or more other antibodies.

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

A neutralizing antibody or an inhibitory antibody is an antibody that inhibits at least one activity of a target—usually a polypeptide—such as by blocking the binding of the polypeptide to a ligand to which it normally binds, or by disrupting or otherwise interfering with a protein-protein interaction of the polypeptide with a second polypeptide. An activating antibody is an antibody that increases an activity of a polypeptide. Antibodies may function as mimics of a target protein activity, or as blockers of the target protein activity, with therapeutic effect derived therein.

Also specifically contemplated are human antibodies (arising from human genes) and humanized antibodies, either of which are suitable for administration to humans without engendering an adverse immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following methods known in the art, such as by substituting rodent CDRs or CDR sequences for the corresponding sequences of a human antibody (see, for instance, U.S. Pat. No. 5,225,539; Jones et al., Nature 321(6069):522-525, 1986; Riechmann et al., J Mol Biol. 203(3):825-828, 1988; Verhoeyen et al., Science 239(4847):1534-1536, 1988; Riechmann et al., Nature 332(6162):323-327 1988; or Verhoeyen et al., Bioessays 8(2):74-78, 1988).

Antibodies can also be linked to compounds that facilitate quantification of a particular antigen, for example using the TaqMan™ Protein Assay system (as described by Swartzman et al., Methods 50(4):S23-6, 2010).

Antisense compound: Refers to an oligomeric compound that is at least partially complementary to the region of a target nucleic acid molecule to which it hybridizes. As used herein, an antisense compound that is “specific for” a target nucleic acid molecule is one which specifically hybridizes with and modulates expression of the target nucleic acid molecule. As used herein, a “target” nucleic acid is a nucleic acid molecule to which an antisense compound is designed to specifically hybridize and modulate expression. In one embodiment, the target nucleic acid molecule is PARP (including PARP-1 and PARP-2).

Nonlimiting examples of antisense compounds include primers, probes, antisense oligonucleotides, siRNAs, miRNAs, shRNAs and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self complementarity to allow for hybridization and formation of a fully or partially double-stranded compound.

Antisense oligonucleotide: As used herein, an “antisense oligonucleotide” is a single-stranded antisense compound that is a nucleic acid-based oligomer. An antisense oligonucleotide can include one or more chemical modifications to the sugar, base, and/or internucleoside linkages. Generally, antisense oligonucleotides are “DNA-like” such that when the antisense oligonucleotide hybridizes to a target mRNA, the duplex is recognized by RNase H (an enzyme that recognizes DNA:RNA duplexes), resulting in cleavage of the mRNA.

Base excision repair (BER): Refers to the cellular mechanism that repairs damaged DNA throughout the cell cycle. It is primarily responsible for removing small, non-helix distorting base lesions from the genome. The related nucleotide excision repair pathway repairs bulky helix-distorting lesions. BER is important for removing damaged bases that could otherwise cause mutations by mispairing or lead to breaks in DNA during replication. BER is initiated by DNA glycosylases, which recognize and remove specific damaged or inappropriate bases, forming AP sites. These are then cleaved by an AP endonuclease. The resulting single-strand break can then be processed by either short-patch (where a single nucleotide is replaced) or long-patch BER (where 2-10 new nucleotides are synthesized). In the context of the present disclosure, a BER pathway inhibitor is any agent that inhibits the expression or activity of a molecule in the BER pathway.

Biopsy: A sample of tissue obtained from a living subject.

Chemotherapeutic agent: An agent with therapeutic usefulness in the treatment of diseases characterized by abnormal cell growth or hyperplasia. Such diseases include cancer, autoimmune disease as well as diseases characterized by hyperplastic growth such as psoriasis. One of skill in the art can readily identify a chemotherapeutic agent (for instance, see Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2^{nd} ed., © 2000 Churchill Livingstone, Inc; Baltzer L, Berkery R (eds): Oncology Pocket Guide to Chemotherapy, 2nd ed. St. Louis, Mosby-Year Book, 1995; Fischer D S, Knobf M F, Durivage H J (eds): The Cancer Chemotherapy Handbook, 4th ed. St. Louis, Mosby-Year Book, 1993).

Control: A “control” refers to a sample or standard used for comparison with an experimental sample. In some embodiments, the control is a sample obtained from a patient that is not sensitive to TMZ and methoxyamine, or TMZ and a PARP inhibitor. In some embodiments, the control is a historical control or standard value (i.e. a previously tested control sample or group of samples that represent baseline or normal values that represent a lack of sensitivity to TMZ and methoxyamine, or TMZ and a PARP inhibitor).

Differential expression (or increase or decrease in expression): A difference, such as an increase or decrease, in the conversion of the information encoded in a gene (such as Polβ or MPG) into messenger RNA, the conversion of mRNA to a protein, or both. In some examples, the difference is relative to a control or reference value (or range of values), such as the average expression value of a gene from a group of samples. The difference can also be relative to a control sample or a control subject. Detecting differential expression, or detecting an increase or decrease in expression, can include measuring the quantity of a particular mRNA or protein in an experimental sample and comparing the quantity of the same mRNA protein in a control sample or to a reference value. Quantification can be either numerical or relative.

In some embodiments, the increase or decrease in expression is an increase or decrease in the level of mRNA or protein of at least two-fold compared with the control. In some embodiments, the increase or decrease in expression is of a diagnostically significant amount, which refers to a change of a sufficient magnitude to provide a statistical probability of the diagnosis (i.e. the determination of sensitivity or lack of sensitivity to TMZ and MX, or TMZ and the PARP inhibitor).

DNA repair: A collection of processes by which a cell identifies and corrects damage to the DNA molecules that encode its genome. In human cells, both normal metabolic activities and environmental factors such as UV light can cause DNA damage, resulting in as many as 1 million individual molecular lesions per cell per day. Many of these lesions cause structural damage to the DNA molecule and can alter or eliminate the cell's ability to transcribe the gene that the affected DNA encodes. Other lesions induce potentially harmful mutations in the cell's genome. Consequently, the DNA repair process must be constantly active so it can respond rapidly to any damage in the DNA structure.

The rate of DNA repair is dependent on many factors, including the cell type, the age of the cell, and the extracellular environment. A cell that has accumulated a large amount of DNA damage, or one that no longer effectively repairs damage incurred to its DNA, can enter one of three possible states: an irreversible state of dormancy, known as senescence; apoptosis or programmed cell death or unregulated cell division, which can lead to the formation of a tumor that is cancerous.

Expression: The translation of a nucleic acid into a protein. Proteins may be expressed and remain intracellular, become a component of the cell surface membrane, or be secreted into the extracellular matrix or medium.

Functional activity (of Polβ and MPG): As used herein, “functional activity of Polβ and MPG” refers to the catalytic activity of the proteins. For example, a functional activity of Polβ is DNA polymerase activity or its 5′ dRP lyase activity and a functional activity of MPG is DNA glycosylase activity. Methods of measuring functional activities of proteins, including DNA polymerase and DNA glycosylase activities, are well known and within the capabilities of one of skill in the art.

Hybridization: Oligonucleotides and their analogs hybridize by hydrogen bonding, which includes Watson-Crick, Hoogsteen or reversed Hoogsteen hydrogen bonding, between complementary bases. Generally, nucleic acid consists of nitrogenous bases that are either pyrimidines (cytosine (C), uracil (U), and thymine (T)) or purines (adenine (A) and guanine (G)). These nitrogenous bases form hydrogen bonds between a pyrimidine and a purine, and the bonding of the pyrimidine to the purine is referred to as “base pairing.” More specifically, A will hydrogen bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms that indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions where specific binding is desired, for example under physiological conditions in the case of in vivo assays or systems. Such binding is referred to as specific hybridization.

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization, though waste times also influence stringency. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference.

For purposes of the present disclosure, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize.

Hyperproliferative disease: A disease or disorder characterized by the uncontrolled proliferation of cells. Hyperproliferative diseases include, but are not limited to malignant and non-malignant tumors and psoriasis.

Inhibit expression or activity: As used herein, a compound that inhibits expression or activity of PARP is a compound that reduces the level of PARP mRNA or protein in a cell or tissue, or reduces (including eliminates) one or more activities of PARP. For example, an antisense compound targeting PARP inhibits expression of PARP by promoting the degradation of PARP mRNA, thereby reducing the level of PARP protein. In some embodiments, PARP expression is inhibited at least 10%, at least 25%, at least 50%, at least 75%, at least 90%, or at least 95% relative to a control, such as untreated control cells. As another example, an antibody or small molecule that specifically binds or targets PARP may inhibit a functional activity of PARP, such as PARP catalytic activity (i.e. poly(ADP-ribosylation)). In some embodiments, PARP activity is inhibited at least 10%, at least 25%, at least 50%, at least 75%, at least 90%, or at least 95% relative to a control.

Inhibitor: Any chemical compound, nucleic acid molecule or peptide (such as an antibody), specific for a nucleic acid molecule or gene product that can reduce activity of the gene product or directly interfere with expression of a gene (such as PARP). An inhibitor of the disclosure, for example, can inhibit the activity of a protein that is encoded by the gene either directly or indirectly. Direct inhibition can be accomplished, for example, by binding to a protein and thereby preventing the protein from binding an intended target, such as a receptor. Indirect inhibition can be accomplished, for example, by binding to a protein's intended target, such as a receptor or binding partner, thereby blocking or reducing activity of the protein. Furthermore, an inhibitor of the disclosure can inhibit a gene by reducing or inhibiting expression of the gene, inter alia by interfering with gene expression (transcription, processing, translation, post-translational modification), for example, by interfering with the gene's mRNA and blocking translation of the gene product or by post-translational modification of a gene product, or by causing changes in intracellular localization.

Isolated: An “isolated” biological component, such as a nucleic acid, protein or organelle that has been substantially separated or purified away from other biological components in the environment (such as a cell) in which the component naturally occurs, i.e., other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Methoxyamine (MX): A bioavailable small molecule (CH₃ONH₂) inhibitor delivered intravenously that specifically inhibits BER (Rosa et al., Nucleic Acids Res. 19:5569-5574, 1991). It is currently under a phase I clinical trial under the name of TRC102 (Tracon Pharma, Inc). Methoxyamine inhibits repair of AP sites by binding and modifying the DNA substrate, AP sites, rather than directly inhibiting the enzyme APEL Methoxyamine covalently binds to apurinic/apyrimidinic (AP) DNA damage sites and inhibits base excision repair, which may result in an increase in DNA strand breaks and apoptosis. This agent is thought to potentiate the anti-tumor activity of alkylating agents. MX is also known as O-methylhydroxylamine and methoxylamine.

MicroRNA (miRNA): Single-stranded RNA molecules that regulate gene expression. miRNAs are generally 21-23 nucleotides in length. miRNAs are processed from primary transcripts known as pri-miRNA to short stem-loop structures called pre-miRNA and finally to functional miRNA. Mature miRNA molecules are partially complementary to one or more messenger RNA molecules, and their primary function is to down-regulate gene expression. MicroRNAs regulate gene expression through the RNAi pathway.

N-methylpurine DNA glycosylase (MPG): A DNA glycosylase that initiates repair of DNA base damage (such as TMZ-induced base damage) by the base excision repair pathway. MPG recognizes and removes damaged bases. MPG is also known as alkyladenine DNA glycosylase (AAG).

Neoplasia, malignancy, cancer and tumor: A neoplasm is an abnormal growth of tissue or cells that results from excessive cell division. Neoplastic growth can produce a tumor. The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Malignant tumors are also referred to as “cancer.”

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. In some cases, lymphomas are considered solid tumors.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include fibrosarcoma, myxosarcoma, liposarcoma, chondrosarcoma, osteosarcoma, and other sarcomas, synovioma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, human papilloma virus (HPV)-infected neoplasias, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, melanoma, and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, menangioma, neuroblastoma, retinoblastoma and brain metastasis).

Patient: As used herein, the term “patient” includes human and non-human animals. The preferred patient for treatment is a human. “Patient” and “subject” are used interchangeably herein.

Percent identity: The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. USA 85:2444, 1988; Higgins and Sharp, Gene 73:237-244, 1988; Higgins and Sharp, CABIOS 5:151-153, 1989; Corpet et al., Nucleic Acids Res. 16:10881-10890, 1988; Pearson and Lipman, Proc. Natl. Acad. Sci. USA 85:2444, 1988; and Altschul et al., Nature Genet. 6:119-129, 1994. The NCBI Basic Local Alignment Search Tool (BLAST™) (Altschul et al., J. Mol. Biol. 215:403-410, 1990) is available from several sources, including the National Center for Biotechnology Information (NCBI, Bethesda, Md.) and on the Internet, for use in connection with the sequence analysis programs blastp, blastn, blastx, tblastn and tblastx.

Pharmaceutical agent: A chemical compound or composition capable of inducing a desired therapeutic or prophylactic effect when properly administered to a subject or a cell.

Pharmaceutically acceptable carriers: The pharmaceutically acceptable carriers of use are conventional. Remington's Pharmaceutical Sciences, by E.W. Martin, Mack Publishing Co., Easton, Pa., 15th Edition, 1975, describes compositions and formulations suitable for pharmaceutical delivery of the compositions disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (such as powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Poly(ADP-ribose) polymerase (PARP): As used herein, the term “PARP” includes PARP-1 and PARP-2. PARP-1 is a chromatin-associated enzyme which modifies various nuclear proteins by poly(ADP-ribosyl)ation. The modification is dependent on DNA and is involved in the regulation of various important cellular processes such as differentiation, proliferation, and tumor transformation and also in the regulation of the molecular events involved in the recovery of a cell from DNA damage. PARP-2 contains a catalytic domain and is capable of catalyzing a poly(ADP-ribosyl)ation reaction. This protein has a catalytic domain that is homologous to that of PARP-1, but lacks an N-terminal DNA binding domain which activates the C-terminal catalytic domain of PARP. The basic residues within the N-terminal region of this protein may bear potential DNA-binding properties, and may be involved in the nuclear and/or nucleolar targeting of the protein.

Polymerase β (Polβ): A DNA polymerase involved in base excision repair.

Preventing, treating or ameliorating a disease: “Preventing” a disease refers to inhibiting the full development of a disease. “Treating” refers to a therapeutic intervention that ameliorates a sign or symptom of a disease or pathological condition after it has begun to develop. “Ameliorating” refers to the reduction in the number or severity of signs or symptoms of a disease.

Ribozyme: A catalytic RNA molecule. In some cases, ribozymes can bind to specific sites on other RNA molecules and catalyze the hydrolysis of phosphodiester bonds in the RNA molecules.

RNA interference (RNAi): Refers to a cellular process that inhibits expression of genes, including cellular and viral genes. RNAi is a form of antisense-mediated gene silencing involving the introduction of double stranded RNA-like oligonucleotides leading to the sequence-specific reduction of RNA transcripts. Double-stranded RNA molecules that inhibit gene expression through the RNAi pathway include siRNAs, miRNAs, and shRNAs.

Sample or biological sample: A biological specimen containing genomic DNA, RNA (including mRNA and microRNA), protein, or combinations thereof, obtained from a subject. Examples include, but are not limited to, saliva, peripheral blood, urine, tissue biopsy, surgical specimen, and autopsy material. In some embodiments, the sample is a tumor biopsy from a subject with cancer.

Sensitive: As used herein, a subject that is “sensitive” to TMZ and methoxyamine, or TMZ and a PARP inhibitor is a subject that responds to combination therapy with TMZ and methoxyamine, or TMZ and a PARP inhibitor. Sensitivity to the combination of agents is indicated by, for example, a reduction in tumor volume, growth or metastasis, an increase in cell death of tumor cells, and/or a reduction in one or more symptoms of disease (such as cancer).

Small interfering RNA (siRNA): A double-stranded nucleic acid molecule that modulates gene expression through the RNAi pathway. siRNA molecules are generally 20-25 nucleotides in length with 2-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs.”

Small molecule: A molecule, typically with a molecular weight less than about 1000 Daltons, or in some embodiments, less than about 500 Daltons, wherein the molecule is capable of modulating, to some measurable extent, an activity of a target molecule.

Subject: Living multi-cellular vertebrate organisms, a category that includes both human and veterinary subjects, including human and non-human mammals.

Temozolomide (TMZ): A chemotherapeutic agent that is used to treat certain types of tumors (such as brain tumors, including anaplastic astrocytoma and glioblastoma). TMZ belongs to the family of drugs called alkylating agents. TMZ causes cancer cell cytotoxicity by methylating genomic DNA. TMZ is also known as TEMODAR™.

Therapeutic or therapy: A generic term that includes both diagnosis and treatment. Treatment refers to a prescribed course of action (including administration of therapeutic agents) to alter the normal course of a disorder.

Therapeutically effective amount: A quantity of a specific substance sufficient to achieve a desired effect in a subject being treated.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. Hence “comprising A or B” means including A, or B, or A and B. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### III. Overview of Several Embodiments

TMZ is the preferred chemotherapeutic agent in the treatment of glioma following surgical resection and/or radiation. Resistance to TMZ is attributed to efficient repair and/or tolerance of TMZ-induced DNA lesions. The majority of TMZ-induced DNA base adducts are repaired by the BER pathway and modulation of this pathway can enhance drug sensitivity. MPG initiates BER by removing TMZ-induced N3-methyladenine and N7-methylguanine base lesions, leaving abasic sites (AP sites) in DNA for further processing by BER.

Disclosed herein is the finding that potentiation of TMZ via BER inhibition (such as by using methoxyamine, PARP inhibitors or depletion/knockdown of PARG) is greatly enhanced by over-expression of the BER initiating enzyme MPG. It is also disclosed that methoxyamine-induced potentiation of TMZ in MPG expressing cells is abrogated by elevated-expression of the rate-limiting BER enzyme DNA polymerase B (Polβ), indicating that cells proficient for BER readily repair AP sites in the presence of methoxyamine. It is further disclosed that depletion of Polβ increases PARP inhibitor-induced potentiation in MPG over-expressing cells, indicating that expression of Polβ modulates the cytotoxic effect of combining increased repair initiation and BER inhibition. The studies disclosed herein demonstrate that MPG over-expression, together with inhibition of BER, sensitizes cancer cells to the alkylating agent TMZ in a Polβ-dependent manner. Accordingly, it is proposed herein that the expression level of both MPG and Polβ can be used to predict the effectiveness of MX and PARP-mediated potentiation of TMZ in cancer treatment.

Described herein is the finding that Polβ and MPG can be used as biomarkers to evaluate the sensitivity of a subject to combination therapy that includes treatment with either TMZ and methoxyamine, or TMZ and a PARP inhibitor.

Accordingly, provided herein is a method of determining if a subject will be sensitive to TMZ and methoxyamine, or TMZ and a PARP inhibitor. In some embodiments, the method includes measuring expression of Polβ and MPG in a sample from the subject; and comparing expression of Polβ and MPG in the sample to a control. A decrease in expression of Polβ and an increase in expression of MPG relative to the control indicates the subject is sensitive to TMZ and methoxyamine, or sensitive to TMZ and the PARP inhibitor.

In some embodiments, the method includes determining if the subject will be sensitive to TMZ and methoxyamine. In other embodiments, the method includes determining if the subject will be sensitive to TMZ and a PARP inhibitor.

In some embodiments, the subject has cancer. The cancer can be any type of cancer for which treatment with a chemotherapeutic agent, particularly an alkylating chemotherapeutic agent such as TMZ, is contemplated. The cancer can be, for example, a solid tumor or a hematogenous cancer. In some examples, the solid tumor is a central nervous system (CNS) tumor, a lymphoma, melanoma, osteosarcoma, colorectal cancer, lung cancer, ovarian cancer, breast cancer or an HPV-infected neoplasia. The CNS tumor can be, for example, a glioma, medulloblastoma, astrocytoma, germinoma, meningioma, oligodendroglioma, Schwannoma, craniopharyngioma, ependymoma or CNS lymphoma. In some examples, the hematologic cancer is a leukemia or a lymphoma. However, the above list of cancers is not intended to be limiting.

The PARP inhibitor can be any agent that inhibits expression and/or activity of PARP. In some embodiments, the PARP inhibitor is a small molecule inhibitor, antisense molecule or antibody. In some examples, the antisense molecule is an antisense oligonucleotide, siRNA, miRNA or ribozyme specific for PARP. In some examples, the small molecule inhibitor of PARP is PJ34, ABT-888, AG14699, AG14361, CEP-6800, CEP-8983, INO-1001, KU59436, BSI-201, GPI 21016, GPI15427 or AZD2281. However, other inhibitors of PARP are known and can be used with the disclosed methods.

In some embodiments, the sample obtained from the subject includes cancer/tumor cells, such as from a tumor biopsy.

Measuring expression of Polβ and MPG can be accomplished using any technique known in the art. In some embodiments, measuring expression of Polβ and MPG comprises measuring the level of Polβ and MPG mRNA. In some examples, the level of Polβ and MPG mRNA is measured by RT-PCR. Other methods of detecting and quantifying Polβ and MPG mRNA are contemplated, including in situ hybridization. In some embodiments, measuring expression of Polβ and MPG comprises measuring the level of Polβ and MPG protein. In some examples, the level of Polβ and MPG protein is measured by immunoblot, radioimmunoassay or ELISA, or any other technique that utilizes Polβ- and MPG-specific antibodies in quantitative assays. In other embodiments, measuring expression of Polβ and MPG comprises measuring functional activity of Polβ and MPG. For example, the catalytic activity of the proteins can be measured using an appropriate assay to detect DNA polymerase activity of Polβ and an appropriate assay to detect DNA glycosylase activity of MPG.

The disclosed methods include comparing the expression level of Polβ and MPG to a control. The control can be any suitable control that represents a lack of sensitivity to the combination of TMZ/MX or TMZ/PARP inhibitor. In some examples, the control is a sample obtained from a subject that is not sensitive to TMZ and methoxyamine, or TMZ and a PARP inhibitor. In other examples, the control is a reference value (such as a value determined to represent a lack of sensitivity to TMZ/MX or TMZ/PARP inhibitor, which may be based on historical values from patients that are not sensitive to these combination therapies).

In some embodiments, the method further includes providing an appropriate therapy for the subject. In some cases, the appropriate therapy comprises administration of TMZ in combination with methoxyamine. In other cases, the appropriate therapy comprises administration of TMZ in combination with a PARP inhibitor. The appropriate therapy may further include any acceptable therapy for the treatment of the particular cancer being treated, including, but not limited to radiation therapy, surgery, additional chemotherapeutic agents, cancer-specific antibodies, estrogen receptor inhibitors, EGF receptor inhibitors, kinase inhibitors, histone deacetylase inhibitors, anti-angiogenesis agents and/or cancer vaccines.

In some embodiments, the subject is determined to be sensitive to TMZ and methoxyamine, or TMZ and a PARP inhibitor, and the method further comprises administering TMZ in combination with methoxyamine, or TMZ in combination with the PARP inhibitor, to the subject. In some examples, the subject is determined to be sensitive to TMZ and methoxyamine, and the method further comprises administering TMZ in combination with methoxyamine to the subject. In other examples, the subject is determined to be sensitive to TMZ and a PARP inhibitor, and the method further comprises administering TMZ in combination with the PARP inhibitor to the subject.

Further provided herein is a method of determining if a subject will be sensitive to TMZ and BER pathway inhibitor. In some embodiments, the method includes measuring expression of Polβ and MPG in a sample from the subject; and comparing expression of Polβ and MPG in the sample to a control. A decrease in expression of Polβ and an increase in expression of MPG relative to the control indicates the subject is sensitive to TMZ and the BER pathway inhibitor.

### IV. Methods of Detecting Expression

As described below, expression of one or more genes (such as Polβ or MPG) can be detected using any one of a number of methods well known in the art. Expression of either mRNA or protein is contemplated herein.

A. Methods for Detection of mRNA

Gene expression can be evaluated by detecting mRNA encoding the gene of interest. Thus, the disclosed methods can include evaluating mRNA encoding Polβ and MPG. In some examples, the mRNA is quantified.

RNA can be isolated from a sample from a subject, such as from a tumor biopsy, using methods well known to one skilled in the art, including commercially available kits. General methods for mRNA extraction are well known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56:A67 (1987), and De Andres et al., BioTechniques 18:42044 (1995). In one example, RNA isolation can be performed using purification kit, buffer set and protease from commercial manufacturers, such as QIAGEN®, according to the manufacturer's instructions. For example, total RNA from cells in culture (such as those obtained from a subject) can be isolated using QIAGEN® RNeasy mini-columns. Other commercially available RNA isolation kits include MASTERPURE®, Complete DNA and RNA Purification Kit (EPICENTRE® Madison, Wis.), and Paraffin Block RNA Isolation Kit (Ambion, Inc.). Total RNA from tissue samples can be isolated using RNA Stat-60 (Tel-Test). RNA prepared from tumor or other biological samples can be isolated, for example, by cesium chloride density gradient centrifugation.

Methods of gene expression profiling include methods based on hybridization analysis of polynucleotides, methods based on sequencing of polynucleotides, and proteomics-based methods. In some examples, mRNA expression in a sample is quantified using northern blotting or in situ hybridization (Parker & Barnes, Methods in Molecular Biology 106:247-283, 1999); RNAse protection assays (Hod, Biotechniques 13:852-4, 1992); and PCR-based methods, such as reverse transcription polymerase chain reaction (RT-PCR) (Weis et al., Trends in Genetics 8:263-4, 1992). Alternatively, antibodies can be employed that can recognize specific duplexes, including DNA duplexes, RNA duplexes, and DNA-RNA hybrid duplexes or DNA-protein duplexes. Representative methods for sequencing-based gene expression analysis include Serial Analysis of Gene Expression (SAGE), and gene expression analysis by massively parallel signature sequencing (MPSS). In one example, RT-PCR can be used to compare mRNA levels in different samples, in normal and tumor tissues, with or without drug treatment, to characterize patterns of gene expression, to discriminate between closely related mRNAs, and to analyze RNA structure.

Methods for quantifying mRNA are well known in the art. In some examples, the method utilizes RT-PCR. Generally, the first step in gene expression profiling by RT-PCR is the reverse transcription of the RNA template into cDNA, followed by its exponential amplification in a PCR reaction. Two commonly used reverse transcriptases are avian myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp™ RNA PCR kit (Perkin Elmer, CA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TAQMAN® RT-PCR can be performed using commercially available equipment, such as, for example, ABI PRISM 7700® Sequence Detection System®(Perkin-Elmer-Applied Biosystems, Foster City, Calif.), or Lightcycler (Roche Molecular Biochemicals, Mannheim, Germany). In one example, the 5′ nuclease procedure is run on a real-time quantitative PCR device such as the ABI PRISM 7700® Sequence Detection System®. The system includes of thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and is unaffected by the experimental treatment. RNAs commonly used to normalize patterns of gene expression are mRNAs for the housekeeping genes glyceraldehyde-3-phosphate-dehydrogenase (GAPDH), beta-actin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Held et al., Genome Research 6:986 994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are available from PE Applied Biosystems, 850 Lincoln Centre Drive, Foster City, Calif. 94404 under the trademark ABI PRISM® 7700.

The steps of a representative protocol for quantifying gene expression using fixed, paraffin-embedded tissues as the RNA source, including mRNA isolation, purification, primer extension and amplification are given in various publications (see Godfrey et al., J. Mol. Diag. 2:84 91, 2000; Specht et al., Am. J. Pathol. 158:419-29, 2001). Briefly, a representative process starts with cutting about 10 μm thick sections of paraffin-embedded tumor tissue samples or adjacent non-cancerous tissue. The RNA is then extracted, and protein and DNA are removed. Alternatively, RNA is located directly from a tumor sample or other tissue sample. After analysis of the RNA concentration, RNA repair and/or amplification steps can be included, if necessary, and RNA is reverse transcribed using gene specific promoters followed by RT-PCR. The primers used for the amplification are selected so as to amplify a unique segment of the gene of interest, such as mRNA encoding Polβ or MPG. In some embodiments, expression of other genes is also detected. Primers that can be used to amplify Polβ or MPG are commercially available or can be designed and synthesized according to well known methods.

An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified DNA from each segment is determined and compared to a standard curve to determine the amount of the target nucleic acid segment that was present in the sample prior to amplification.

In some embodiments of this method, the expression of a “housekeeping” gene or “internal control” can also be evaluated. These terms include any constitutively or globally expressed gene whose presence enables an assessment of Polβ or MPG mRNA levels. Such an assessment includes a determination of the overall constitutive level of gene transcription and a control for variations in RNA recovery.

In situ hybridization (ISH) is another method for detecting and comparing expression of genes of interest. ISH applies and extrapolates the technology of nucleic acid hybridization to the single cell level, and, in combination with the art of cytochemistry, immunocytochemistry and immunohistochemistry, permits the maintenance of morphology and the identification of cellular markers to be maintained and identified, and allows the localization of sequences to specific cells within populations, such as tissues and blood samples. ISH is a type of hybridization that uses a complementary nucleic acid to localize one or more specific nucleic acid sequences in a portion or section of tissue (in situ), or, if the tissue is small enough, in the entire tissue (whole mount ISH). RNA ISH can be used to assay expression patterns in a tissue, such as the expression of Polβ or MPG.

Sample cells or tissues are treated to increase their permeability to allow a probe, such as a Polβ- or MPG-specific probe, to enter the cells. The probe is added to the treated cells, allowed to hybridize at pertinent temperature, and excess probe is washed away. A complementary probe is labeled with a radioactive, fluorescent or antigenic tag, so that the probe's location and quantity in the tissue can be determined using autoradiography, fluorescence microscopy or immunoassay. The sample may be, for example a tumor biopsy. Since the sequences of Polβ and MPG are known, probes can be designed accordingly such that the probes specifically bind the gene of interest.

In situ PCR is the PCR based amplification of the target nucleic acid sequences prior to ISH. For detection of RNA, an intracellular reverse transcription step is introduced to generate complementary DNA from RNA templates prior to in situ PCR. This enables detection of low copy RNA sequences.

Prior to in situ PCR, cells or tissue samples are fixed and permeabilized to preserve morphology and permit access of the PCR reagents to the intracellular sequences to be amplified. PCR amplification of target sequences is next performed either in intact cells held in suspension or directly in cytocentrifuge preparations or tissue sections on glass slides. In the former approach, fixed cells suspended in the PCR reaction mixture are thermally cycled using conventional thermal cyclers. After PCR, the cells are cytocentrifuged onto glass slides with visualization of intracellular PCR products by ISH or immunohistochemistry. In situ PCR on glass slides is performed by overlaying the samples with the PCR mixture under a coverslip which is then sealed to prevent evaporation of the reaction mixture. Thermal cycling is achieved by placing the glass slides either directly on top of the heating block of a conventional or specially designed thermal cycler or by using thermal cycling ovens.

Detection of intracellular PCR products is generally achieved by one of two different techniques, indirect in situ PCR by ISH with PCR-product specific probes, or direct in situ PCR without ISH through direct detection of labeled nucleotides (such as digoxigenin-11-dUTP, fluorescein-dUTP, 3H-CTP or biotin-16-dUTP), which have been incorporated into the PCR products during thermal cycling.

B. Methods for Detection of Protein

In some examples, expression of Polβ and MPG protein is analyzed. Suitable biological samples include, for example, cancerous/tumor cells or a tumor biopsy from a subject. Antibodies specific for Polβ or MPG can be used for detection and quantification by one of a number of immunoassay methods that are well known in the art, such as those presented in Harlow and Lane (Antibodies, A Laboratory Manual, CSHL, New York, 1988). Methods of constructing such antibodies are known in the art.

Any standard immunoassay format (such as ELISA, Western blot/immunoblot, or radioimmunoassay) can be used to measure protein levels. Immunohistochemical techniques can also be utilized for Polβ and MPG protein detection and quantification. General guidance regarding such techniques can be found in Bancroft and Stevens (Theory and Practice of Histological Techniques, Churchill Livingstone, 1982) and Ausubel et al. (Current Protocols in Molecular Biology, John Wiley & Sons, New York, 1998).

For the purposes of quantifying a protein, a biological sample of the subject that includes cellular proteins can be used. Quantification of Polβ and MPG protein can be achieved by immunoassay methods known in the art. The amounts of Polβ and MPG protein in the tumor can be compared to levels of the protein found in cells from a control subject or other control (such as a standard value or reference value). A significant increase or decrease in the amount can be evaluated using statistical methods known in the art.

Quantitative spectroscopic approaches methods, such as SELDI, can be used to analyze protein expression in a sample. For example, surface-enhanced laser desorption-ionization time-of-flight (SELDI-TOF) mass spectrometry can be used to detect protein expression, for example by using the ProteinChip™ (Ciphergen Biosystems, Palo Alto, Calif.). Such methods are well known in the art (for example see U.S. Pat. No. 5,719,060; U.S. Pat. No. 6,897,072; and U.S. Pat. No. 6,881,586). SELDI is a solid phase method for desorption in which the analyte is presented to the energy stream on a surface that enhances analyte capture or desorption.

Briefly, one version of SELDI uses a chromatographic surface with a chemistry that selectively captures analytes of interest. Chromatographic surfaces can be composed of hydrophobic, hydrophilic, ion exchange, immobilized metal, or other chemistries. For example, the surface chemistry can include binding functionalities based on oxygen-dependent, carbon-dependent, sulfur-dependent, and/or nitrogen-dependent means of covalent or noncovalent immobilization of analytes. The activated surfaces are used to covalently immobilize specific “bait” molecules such as antibodies, receptors, or oligonucleotides often used for biomolecular interaction studies such as protein-protein and protein-DNA interactions.

The surface chemistry allows the bound analytes to be retained and unbound materials to be washed away. Subsequently, analytes bound to the surface (such as tumor-associated proteins) can be desorbed and analyzed by any of several means, for example using mass spectrometry. When the analyte is ionized in the process of desorption, such as in laser desorption/ionization mass spectrometry, the detector can be an ion detector. Mass spectrometers generally include means for determining the time-of-flight of desorbed ions. This information is converted to mass. However, one need not determine the mass of desorbed ions to resolve and detect them: the fact that ionized analytes strike the detector at different times provides detection and resolution of them. Alternatively, the analyte can be detectably labeled (for example with a fluorophore or radioactive isotope). In these cases, the detector can be a fluorescence or radioactivity detector. A plurality of detection means can be implemented in series to fully interrogate the analyte components and function associated with retained molecules at each location in the array.

Therefore, in a particular example, the chromatographic surface includes antibodies that specifically bind Polβ and/or MPG. In other examples, the chromatographic surface consists essentially of, or consists of, antibodies that specifically bind Polβ and/or MPG. In some examples, the chromatographic surface includes antibodies that bind other molecules, such as housekeeping proteins (e.g. actin or myosin). In another example, antibodies are immobilized onto the surface using a bacterial Fc binding support. The chromatographic surface is incubated with a sample. The antigens present in the sample can recognize the antibodies on the chromatographic surface. The unbound proteins and mass spectrometric interfering compounds are washed away and the proteins that are retained on the chromatographic surface are analyzed and detected by SELDI-TOF. The MS profile from the sample can be then compared using differential protein expression mapping, whereby relative expression levels of proteins at specific molecular weights are compared by a variety of statistical techniques and bioinformatic software systems.

### V. PARP Inhibitors

Any suitable type of PARP inhibitor is contemplated for use with the disclosed methods. PARP inhibitors include, but are not limited to, small molecule inhibitors, nucleic acid molecules, such as antisense compounds, and proteins, such as PARP-specific antibodies. Any compound that inhibits PARP activity or expression is contemplated. Methods of identifying PARP inhibitors are well known in art and are described below. In addition, a number of PARP inhibitors are already known and have been previously described.

A. Small Molecule Inhibitors of PARP

A number of small molecule inhibitors of PARP are known, particularly for the treatment of cancer. Examples of known small molecule inhibitors of PARP include, but are not limited to, PJ34, ABT-888, AG14699, AG14361, CEP-6800, CEP-8983, INO-1001, KU59436, BSI-201, GPI 21016, GPI15427, KU0058684, KU058948 and AZD2281 (see, for example, Kelly and Fishel, Anticancer Agents Med Chem 8(4):417-425, 2008; Chalmers, Br Med Bull 89:23-40, 2009; Ratnam and Low, Clin Cancer Res 15(5):1383-1388, 2007; McCabe et al., Cancer Biol Ther 4(9):934-936, 2005; Cheng et al., Mol Cancer Ther 4(9):1364-1368, 2005; Miknyoczki et al., Mol Cancer Ther 2:371-382, 2003). The list of small molecule PARP inhibitors provided herein is not intended to be exclusive. Any compound that inhibits PARP expression or activity is contemplated for use with the disclosed methods.

B. Antisense Compounds

Generally, the principle behind antisense technology is that an antisense compound hybridizes to a target nucleic acid and effects the modulation of gene expression activity, or function, such as transcription, translation or splicing. The modulation of gene expression can be achieved by, for example, target RNA degradation or occupancy-based inhibition. An example of modulation of target RNA function by degradation is RNase H-based degradation of the target RNA upon hybridization with a DNA-like antisense compound, such as an antisense oligonucleotide. Antisense oligonucleotides can also be used to modulate gene expression, such as splicing, by occupancy-based inhibition, such as by blocking access to splice sites.

Another example of modulation of gene expression by target degradation is RNA interference (RNAi) using small interfering RNAs (siRNAs). RNAi is a form of antisense-mediated gene silencing involving the introduction of double stranded (ds)RNA-like oligonucleotides leading to the sequence-specific reduction of targeted endogenous mRNA levels. Another type of antisense compound that utilizes the RNAi pathway is a microRNA. MicroRNAs are naturally occurring RNAs involved in the regulation of gene expression. However, these compounds can be synthesized to regulate gene expression via the RNAi pathway. Similarly, shRNAs are RNA molecules that form a tight hairpin turn and can be used to silence gene expression via the RNAi pathway. The shRNA hairpin structure is cleaved by the cellular machinery into siRNA.

Other compounds that are often classified as antisense compounds are ribozymes. Ribozymes are catalytic RNA molecules that can bind to specific sites on other RNA molecules and catalyze the hydrolysis of phosphodiester bonds in the RNA molecules. Ribozymes modulate gene expression by direct cleavage of a target nucleic acid, such as a messenger RNA.

Each of the above-described antisense compounds provides sequence-specific target gene regulation. This sequence-specificity makes antisense compounds effective tools for the selective modulation of a target nucleic acid of interest. In one embodiment, the target nucleic acid is PARP.

Any type of antisense compound that specifically targets and regulates expression of PARP is contemplated for use with the disclosed methods. Such antisense compounds include single-stranded compounds, such as antisense oligonucleotides, and double-stranded compounds, including compounds with at least partial double-stranded structure, including siRNAs, miRNAs, shRNAs and ribozymes. In some embodiments, PARP expression is inhibited at least 10%, at least 25%, at least 50%, at least 75%, at least 90%, or at least 95% relative to a control.

Methods of designing, preparing and using antisense compounds that specifically target PARP are within the abilities of one of skill in the art. Furthermore, sequences for PARP are publicly available.

Antisense compounds specifically targeting PARP can be prepared by designing compounds that are complementary to a PARP nucleotide sequence, particularly the PARP mRNA sequence. Antisense compounds targeting PARP need not be 100% complementary to PARP to specifically hybridize and regulate expression the target gene. For example, the antisense compound, or antisense strand of the compound If a double-stranded compound, can be at least 75%, at least 80%, at least 85%, at least 90%, at least 95%, at least 99% or 100% complementary to the selected PARP nucleic acid sequence. Methods of screening antisense compounds for specificity are well known in the art (see, for example, U.S. Pre-Grant Publication No. 2003-0228689).

In some examples, the antisense compounds contain one or more modifications to enhance nuclease resistance and/or increase activity of the compound. Modified antisense compounds include those comprising modified bases, modified sugars, modified backbones or non-natural internucleoside linkages. Preparation and use of modified antisense compounds is well known in the art (see, for example, U.S. Patent Application Publication No. 2003-0228689).

Antisense compounds can be delivered to a cell, tissue or organ using any of a number of methods well known in the art. Such methods include, but are not limited to, liposomal-mediated transfection, electroporation and conjugation of the antisense compound to a cell-penetrating peptide. Transfection of antisense compounds generally involves the use of liposomal-mediated transfection reagents, a number of which are commercially available. Methods for transfection and electroporation of nucleic acids, including antisense compounds, are well known in the art (see, for example, U.S. Pat. Nos. 7,307,069 and 7,288,530; Pretchtel et al., J. Immunol. Methods 311(1-2):139-52, 2006; Ghartey-Tagoe et al., Int. J. Pharm. 315(1-2):122-133, 2006). Antisense compounds are administered to a subject in any suitable manner, preferably with pharmaceutically acceptable carriers. Pharmaceutically acceptable carriers are determined in part by the particular composition being administered, as well as by the particular method used to administer the composition. Accordingly, there is a wide variety of suitable formulations of pharmaceutical compositions of the present disclosure, which are discussed in further detail below.

C. Antibodies Specific for PARP

A PARP polypeptide or a fragment or conservative variant thereof can be used to produce antibodies which are immunoreactive or specifically bind to an epitope of PARP. Polyclonal antibodies, antibodies which consist essentially of pooled monoclonal antibodies with different epitopic specificities, as well as distinct monoclonal antibody preparations are included.

The preparation of polyclonal antibodies is well known to those skilled in the art (see, for example, Green et al., “Production of Polyclonal Antisera,” in: Immunochemical Protocols, pages 1-5, Manson, ed., Humana Press, 1992; Coligan et al., “Production of Polyclonal Antisera in Rabbits, Rats, Mice and Hamsters,” in: Current Protocols in Immunology, section 2.4.1, 1992).

The preparation of monoclonal antibodies likewise is conventional (see, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988). Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, e.g., Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992).

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, such as syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies can also be derived from a subhuman primate antibody. General techniques for raising therapeutically useful antibodies in baboons can be found, for example, in PCT Publication No. WO 91/11465, 1991; and Losman et al., Int. J. Cancer 46:310, 1990.

Alternatively, an antibody that specifically binds a PARP polypeptide can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Natl. Acad. Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from STRATAGENE Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol. 6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (SCA), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Natl. Acad. Sci. U.S.A. 69:2659, 1972). Alternatively, the variable chains can be linked by an intermolecular disulfide bond or cross-linked by chemicals such as glutaraldehyde (see, for example, Sandhu, Crit. Rev. Biotech. 12:437, 1992). Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Antibodies can be prepared using an intact polypeptide or fragments containing small peptides of interest as the immunizing antigen. The polypeptide or a peptide used to immunize an animal can be derived from substantially purified polypeptide produced in host cells, in vitro translated cDNA, or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see, for example, Coligan et al., Unit 9, Current Protocols in Immunology, Wiley Interscience, 1991).

D. Administration of PARP Inhibitors

PARP inhibitors are preferably administered to a subject in a pharmaceutically acceptable carrier or diluent. The choice of pharmaceutically acceptable carrier will depend on a variety of factors, including the type of inhibitor, route of administration, and the disease to be treated. An inhibitor of PARP can be administered using any suitable route, including, for example, parenteral, oral or topical.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Administration can be accomplished by single or multiple doses. The dose required will vary from subject to subject depending on the species, age, weight and general condition of the subject, the particular type of PARP inhibitor being used (for example, small molecule, antisense compound or antibody) and its mode of administration. An appropriate dose can be determined by one of ordinary skill in the art using only routine experimentation. If administered in multiple doses, the time between delivery of each dose can vary between days, weeks, months and years.

The following examples are provided to illustrate certain particular features and/or embodiments. These examples should not be construed to limit the disclosure to the particular features or embodiments described.

### EXAMPLE

### Example 1

### Materials and Methods

### Chemicals and Reagents

Alpha EMEM was from Mediatech. Fetal bovine serum (FBS), heat inactivated FBS, Pen/Strep/Ampho, glutamine and antibiotic/antimycotic were from Invitrogen (Carlsbad, Calif.). Temozolomide (NSC#362856; IUPAC name: 3-methyl-2-oxo-1,3,4, 5,8-pentazabicyclo[4.3.0]nona-4,6,8-triene-7-carbooxamide; CAS number: 856622-93-1) (Taioli et al., BMC Cancer, 9:354, 2009) was obtained from the National Cancer Institute Developmental Therapeutics Program (Bethesda, Md.). A temozolomide (TMZ) stock solution was prepared in DMSO at 100 mM. Puromycin, Gentamicin and Neomycin were purchased from Clontech Laboratories (Mountain View, Calif.), Irvine Scientific (Santa Ana, Calif.) and Invitrogen (Carlsbad, Calif.), respectively. PJ34 and methoxyamine hydrochloride was purchased from Calbiochem (Gibbstown, N.J.) and Sigma (St. Louis, Mo.), respectively. ABT888 was provided by Abbott Laboratories (Abbott Park, Ill.).

### Plasmid Expression and RNAi Vectors

Human MPG (WT) was expressed using the plasmid pRS 1422, as described previously (Trivedi et al., Mol. Pharmacol., 2008). The MPG expression plasmid (pRS 1422) was then mutated at residue N169 using the Quickchange XL Site-Directed Mutagenesis Kit (Stratagene, La Jolla, Calif.) to yield pIRES-Neo-MPG-N169D. The construction of mammalian expression plasmids of FLAG-tagged human WT and mutant Polβ (K72A) has been described previously (Tang et al., Mol. Cancer Res. 8(1):67-79, 2010). The shuttle vectors (control: pLKO.1-puro-TurboGFP; PARG: pCMV-tGFP-PARG) of HIV-based lentiviral shRNA expression system were purchased from Sigma (St. Louis, Mo.). Lentiviruses expressing PARG specific or control shRNA were prepared by the UPCI Lentiviral core facility (Pittsburgh, Pa.). The shRNA target sequences for PARG are described in detail in Table 1.

TABLE 1
MISSION ™ TRC shRNA Target Set for PARG

| | | ShRNA |
|---|---|---|
| TRC No. | Sequence Information | # |
| TRCN0000051303 | CCGGGCTAAGATGAAATCGGAGTATCTCGAGATACT | 1 |
| | CCGATTTCATCTTAGCTTTTTG (SEQ ID NO: 9) | |
| | Clone ID: NM_003631.1-2105s1c1 | |
| | Accession Number(s): NM_003631.2 | |
| TRCN0000051306 | CCGGCGATTGCATGTCACTTACGAACTCGAGTTCGTA | 2 |
| | AGTGACATGCAATCGTTTTTG (SEQ ID NO: 10) | |
| | Clone ID: NM_003631.1-2315s1c1 | |
| | Accession Number(s): NM_003631.2 | |
| | Region: CDS | |
| TRCN0000051307 | CCGGGCCTAGGAAATTCTCCTCCATCTCGAGATGGAG | 3 |
| | GAGAATTTCCTAGGCTTTTTG (SEQ ID NO: 11) | |
| | Clone ID: NM_003631.1-1026s1c1 | |
| | Accession Number(s): NM_003631.2 | |
| | Region: CDS | |
| TRCN0000051305 | CCGGGCTGAGCGAGATGTGGTTTATCTCGAGATAAAC | 4 |
| | CACATCTCGCTCAGCTTTTTG (SEQ ID NO: 12) | |
| | Clone ID: NM_003631.1-2843s1c1 | |
| | Accession Number(s): NM_003631.2, XM_937616.2 | |
| | Region: CDS | |
| TRCN0000051304 | CCGGGCAGTTTAGTAATGCTAACATCTCGAGATGTTA | 5 |
| | GCATTACTAAACTGCTTTTTG (SEQ ID NO: 13) | |
| | Clone ID: NM_003631.1-706s1c1 | |
| | Accession Number(s): NM_003631.2 | |
| | Region: CDS | |

### Cell Culture and Cell Line Development

The glioblastoma cell line LN428 was cultured in Alpha EMEM supplemented with 10% heat inactivated FBS, glutamine, antibiotic/antimycotic and Gentamycin. LN428 is an established glioblastoma-derived cell line with mutations in p53, and deletions in p14^{ARF} and p16, but is WT for PTEN (Park et al., Cancer Res 62:6318-6322, 2002; Ishii et al., Brain Pathol 9:469-479, 1999). Additional glioma cell lines used herein are detailed in Table 2. Cells were maintained at 37° C. with 5% CO₂.

TABLE 2
Characteristics of the glioma cell lines

| Cell Line | ATCC # | Tissue | Other details | Citation(s)* |
|---|---|---|---|---|
| LN428 | — | Brain, | Mutations in p53, | Tang et al. |
| | | glioblastoma | deletions in | Park et al. |
| | | | p14^{ARF} and p16, | Ishii et al. |
| | | | WT for PTEN | |
| T98G | CRL-1690 | Brain, | Elevated | Stein et al. |
| | | glioblastoma | expression of | |
| | | | MGMT | |
| A-172 | CRL-1620 | Brain, | — | Giard et al. |
| | | glioblastoma | | |
| DBTRG- | CRL-2020 | Glioblastoma; | — | Kruse et al. |
| 05MG | | brain; glial | | |
| | | cell | | |
| M059K | CRL-2365 | Malignant; | Proficient for | Allalunis- |
| | | brain; | DNA-PK | Turner et al. |
| | | glioblastoma | expression | |
| M059J | CRL-2366 | Malignant; | Deficient for | Allalunis- |
| | | brain; | DNA-PK | Turner et al. |
| | | glioblastoma | expression | |
| U87MG | HTB-14 | Brain, | PTEN null; | Clark et al. |
| | | glioblastoma, | predicted to be | |
| | | astrocytoma | null for MRE11B | |
| | | | and XPC | |
| LN215 | — | Brain, | — | Van et al. |
| | | glioblastoma | | Van Meir |
| | | | | et al. |
| LN235 | — | Brain, | — | Van et al. |
| | | glioblastoma | | Van Meir |
| | | | | et al. |
| LN319 | — | Brain, | — | Van et al. |
| | | glioblastoma | | Van Meir |
| | | | | et al. |
| LN444 | — | Brain, | — | Van et al. |
| | | glioblastoma | | Van Meir |
| | | | | et al. |

*Citations: Tang et al., Mol Cancer Res 8: 67-79, 2010; Park et al., Cancer Res 62: 6318-6322, 2002; Ishii et al., Brain Pathol 9: 469-479, 1999; Stein et al., J Cell Physiol 99: 43-54, 1979; Giard et al., J Natl Cancer Inst 51: 1417-1423, 1973; Kruse et al., In Vitro Cell Dev Biol 28A: 609-614, 1992; Allalunis-Turner et al., Radiat Res 134: 349-354, 1993; Clark et al., PLoS Genet 6: e1000832, 2010; Van et al., Cancer Res 50: 6683-6688, 1990; Van Meir et al., Cancer Res 54: 649-652, 1994

Human WT MPG, mutant MPG (N169D), WT Flag Polβ and Flag Pol β (K72A) expressing cell lines were developed by transfecting cells with corresponding plasmids using FuGene™ 6 Transfection Reagent (Roche, Indianapolis, Ind.) according to the manufacturer's protocol. Transfected cells were cultured in G418 and/or Puromycin for 2 weeks and individual clones stably expressing human MPG or Pol β were selected. Lentiviral particles were generated by co-transfection of 4 plasmids, pLKO.1-puro-TurboGFP (control, expresses GFP) or pCMV-tGFP-PARG (PARG shRNA, co-expresses PARG shRNA and GFP) together with pMD2.g(VSVG), pVSV-REV and PMDLg/pRRE, into 293-FT cells (Zufferey et al., J. Virol., 72:9873-9880, 1998; Zufferey et al., Nat. Biotechnol., 15:871-875, 1997) using FuGene™ 6 Transfection Reagent (Roche, Indianapolis, Ind.), as described previously (Tang et al., Mol. Cancer Res. 8(1):67-79, 2010). Forty-eight hours after transfection, lentivirus containing supernatant was collected and passed through 0.45 μm filters to isolate the viral particles. Lentiviral transduction was performed as described earlier (Trivedi et al., Mol. Pharmacol., 2008). Briefly, 6.0×10⁴ cells were seeded into 6-well plate 24 hours before transduction. Cells were transduced for 18 hours at 32° C. and cultured for 72 hours at 37° C. Cells expressing copGFP only or both copGFP and PARG specific shRNA were isolated by fluorescence activated cell sorting (FACS).

LN428 cell lines engineered to over-express MGMT (LN428/MGMT) were developed by plasmid transfection. Briefly, 1.5×10⁵ cells were seeded into 60 mm dishes and incubated for 24-30 hours at 5% CO₂ at 37° C. The human MGMT expression plasmid (pIRES-Puro-hMGMT) was transfected using FuGene™ 6 Transfection Reagent (Roche; Indianapolis, Ind.) according to the manufacturer's instructions. Stable cell lines were selected in puromycin (0.5 μg/ml) for 2 weeks, individual clones (stably expressing human MGMT) were expanded and 30 μg of nuclear extract was analyzed by immunoblot analysis for the expression of human MGMT protein.

### Cell Cytotoxicity Assay

Short-term cell survival assay: TMZ or TMZ+MX induced cytotoxicity was determined by an MTS assay, a modified MTT assay as described previously (Trivedi et al., Cancer Res., 65:6394-6400, 2005). Results were calculated from the average of three or four separate experiments and are reported as the percentage of treated cells relative to the cells without treatment (% control). Long term cell survival assay: cells were seeded into 6-well plates 24 hours before exposure to PJ34 (4 μm), ABT-888 (10 μm) or DMSO as control. Thirty minutes later, cells were treated with TMZ alone, TMZ plus PJ34 (2 μm) or TMZ plus ABT-888 (5 μm) for 6 hours. Cells were washed with PBS, trypsinized, resuspended and counted before being re-seeded into three 100 mm cell culture dishes at 8000 cells each. Cells were incubated with or without 2 μm PJ34 or 5 μm ABT-888 for 10 days before being counted. Results were calculated from three independent experiments and reported as percentage relative to the control treatment (% control).

### Cell Extract Preparation and Western Blot

Nuclear extracts were prepared and protein concentration was determined as described previously (Trivedi et al., Cancer Res., 65:6394-6400, 2005; Tang et al., Mol. Cancer Res. 8(1):67-79, 2010). Twenty microgram of protein was loaded on a precast 4-20% NuPAGE™ Tris-Glycine gel (Invitrogen, Carlsbad, Calif.). For whole cell extracts used in probing Poly(ADP-ribose) (PAR), 3×10⁶ cells were seeded into a 100 mm cell culture dish 24 hours before drug treatment. Cells were then treated with TMZ (1.5 mM) or DMSO (control) for different periods of time. After treatment, cells were washed twice with cold PBS, collected and lysed in 400 μL of 2×Laemmli buffer (2% SDS, 20% Glycerol, 62.5 mM Tris-HCl pH6.8, 0.01% Bromophenol Blue). Samples were boiled for 8 minutes and extracts from approximately 1.5×10⁵ cells were loaded each lane on a 4-12% pre-cast NuPAGE™ Tris-Glycine gel (Invitrogen, Carlsbad, Calif.) for immunoblot analysis. The following primary antibodies were used in immunoblot assays: anti-human MPG (Mab; clone 506-3D) (Trivedi et al., Mol. Pharmacol, 2008); anti-Polβ (Mab clone 61; Thermo Fisher Scientific); anti-APE1 (EMD Biosciences); anti-PCNA (Santa Cruz); anti-poly(ADP-ribose) (PAR) (Clone 10H); anti-MGMT (Novus, Littleton, Colo.).

Isolation and Analysis of Total RNA from Normal Brain and GBM Tumor Tissue

Formalin-fixed paraffin-embedded (FFPE) tumor and normal tissue was obtained and evaluated by a board-certified pathologist (RLH) to verify that representative sections were used. All tissue samples were obtained using an honest broker and samples were de-identified. Total cellular RNA was isolated from archival FFPE tumor (GBM) and normal brain tissue using the RecoverAll Total Nucleic Acid isolation kit (Ambion; Austin, Tex.) and the final concentration determined using a NANODROP™ spectrophotometer (Thermo Fisher Scientific; Waltham, Mass.). Following isolation, cDNA was synthesized from 50 ng of RNA using the Applied Biosystems High Capacity cDNA Reverse Transcription Kit (part #4375575), essentially as described previously (Yoshizawa et al., PLUS ONE 4:e6493, 2009). Briefly, cDNA was pre-amplified for 10 cycles using the TaqMan® PreAmp Master Mix (part #4391128) and diluted 1:5. The pre-amplified cDNA was next analyzed using validated Applied Biosystems TaqMan® Gene Expression Assays (human MPG: Hs00357983-G1; human Polβ: Hs01099715-M1; and human PARP1: Hs00911369-G1) and normalized to the expression of human β-actin (part #4333762T). Expression analysis was determined using the ΔΔC_{T} protocol as per the manufacturer to determine the relative level of expression, as compared to human β-actin among all samples. From each tumor sample, expression was normalized to the level of expression in a normal brain sample (sample #20).

### Quantitative RT-PCR Analysis

Expression of MPG, Polβ and PARP1 in the cell lines was measured by quantitative RT-PCR using an Applied Biosystems StepOnePlus™ system as described previously (Tang et al., Mol Cancer Res 8:67-79, 2010). Briefly, 80,000 cells were lysed and reverse transcribed using the Applied Biosystems Taqman® Gene Expression Cells-to-CT™ Kit. Each sample was analyzed in triplicate and the results are an average of all three analyses. Analysis of mRNA expression was conducted as per the manufacturer (ΔΔC_{T} method). Applied Biosystems TaqMan® Gene Expression Assays used were as follows: human MPG: Hs00357983-G1; human Polβ: Hs01099715-M1; and human PARP1: Hs00911369-G1. Each were normalized to the expression of human β-actin (part #4333762T).

### Molecular Beacon MPG Activity Assay

All oligodeoxyribonucleotides were purchased from Integrated DNA Technologies (Coralville, USA) including the following oligonucleotides:

FD-Con, 6-FAM-dGCACTATTGAATTGACACGCCATGTCGATCAATTCAATAGTGC-Dabcyl (SEQ ID NO: 1), where 6-FAM is carboxyfluorescein and Dabcyl is 4-(4′-dimethylaminophenylazo) benzoic acid;
FD-MPG1,6-FAM-dGCACTNTTGAATTGACACGCCATGTCGATCAATTCAATAGTGC-Dabcyl (SEQ ID NO: 2), where N is 3-etho-adenine (8A).

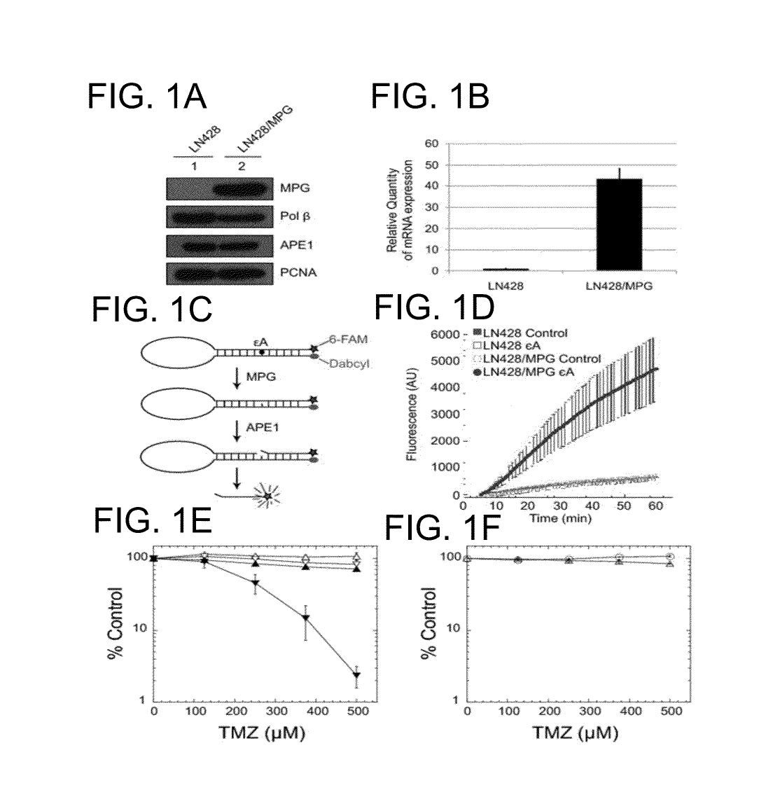

All the oligonucleotides were designed to form a stem-loop structure with 13 nucleotides situated in the loop and 15 base pairs in the stem. Carboxyfluorescein (6-Fam) is a fluorescent molecule that is quenched by Dabcyl in a non-fluorescent manner via Förster Resonance Energy Transfer (FRET) (Clegg, Methods Enzymol 211:353-388, 1992; Yaron et al., Anal Biochem 95:228-235, 1979). Therefore, when the DNA is in a stem-loop structure, the 6-FAM at the 5′ end and the Dabcyl at the 3′ end are brought into close proximity. The close proximity of the 6-FAM to Dabcyl enables efficient quenching of 6-FAM by Dabcyl. If the εA is removed by MPG and the DNA backbone is hydrolyzed by APE1, the 6-FAM-containing oligonucleotide (4 bases in length) will dissociate from the hairpin at 37° C. (FIG. 1C) and the 6-FAM dissociation from the DNA hairpin prevents the quenching by Dabcyl. The increase in 6-FAM-mediated fluorescence is proportional to the amount of εA removed. Any increase in fluorescence in control beacon with a normal adenine would be the result of non-specific cleavage of the DNA backbone.

To ensure the beacons correctly adapted a stem-loop structure, each were incubated at 95° C. for 3 minutes. The beacons were removed from the heat and allowed to slowly cool overnight to room temperature in an insulated container. Once the hairpin was formed no measurable fluorescence was detected and the hairpin was stable at 37° C. for greater than 120 minutes. However, when heated to 95° C., the hairpin unfolds, resulting in maximum fluorescence intensity. Nuclear protein extracts were prepared as described above. Approximately 500 μL of nuclear protein extracts were dialyzed twice using the Slide-A-Lyzer Dialysis Cassette with a 7,000 molecular weight cut-off (Pierce; Rockford, Ill.). The samples were dialyzed for 90 minutes at 4° C. in the following buffer: 50 mM HEPES pH7.5, 100 mM KCl, 0.5 mM EDTA, 20% Glycerol and 1 mM DTT. Reactions were performed using 10 μg of dialyzed protein extract and beacon substrate (final concentration=40 nM) in the following buffer: 25 mM HEPES-KOH pH7.8, 150 mM KCl, 0.5 mM EDTA, 1% Glycerol, 0.5 mM DTT. Fluorescence was measured, every 20 seconds for 60 minutes, using a StepOnePlus™ real-time PCR system and expressed as arbitrary units (AU).

### Molecular Beacon Data Analysis

The fluorescence data was analyzed to enable comparisons across cell lines and for comparison of control and lesion containing BER-beacons. The background fluorescence due to incubation of the beacon alone was eliminated by subtracting the fluorescence values of a control well containing no protein extract from all wells using that molecular beacon. To enable comparisons across different cell lines, molecular beacons and trials, the fluorescence value of the 5-minute time point was selected as the zero value for each well. This value was subtracted from all other time points in that well so all graphs begin from zero AU and 5 minutes after initiating the reaction. Five minutes was selected as the point from which to begin comparisons because time points earlier than four minutes contained variations in absolute fluorescence measurements independent of molecular beacon and cell line. Five minutes was selected to eliminate the variable measurements and to facilitate valid comparisons between trials and conditions. The mean of three separate trials was plotted with error bars representing the standard error of the mean.

### DNA Extraction and MSP Assay for Human MGMT Promoter

DNA was purified from 5×10⁶ LN428 cells and T98G cells using the DNeasy tissue kit (Qiagen, Valencia, Calif.) according to the manufacturer's instruction and methylation of the MGMT promoter was determined by the methylation-specific PCR (MSP). The sense and antisense primers for the methylated human MGMT promoters were 5′TTTCGACGTTCGTAGGTTTTCGC-3′ (SEQ ID NO: 3) and 5′-GCACTCTTCCGAAAACGAAACG-3′ (SEQ ID NO: 4) respectively, and the primers used to detect the unmethylated human MGMT promoters were 5′-TTTGTGTTTTGATGTTTGTAGGTTTTTGT-3′ (SEQ ID NO: 5) and 5′-AACTCCACACTCTTCCAAAAACAAAACA-3′ (SEQ ID NO: 6) respectively (Taioli et al., BMC Cancer, 9:354, 2009). PCR products (93 by for unmethylated human MGMT promoters and 81 by for methylated human MGMT promoters) were analyzed by 4% agarose gel electrophoresis (Invitrogen-Gibco, Carlsbad, Calif.) using Universal unmethylated DNA (Chemicon International, Temecula, Calif.) as a negative control DNA and Universal methylated DNA (Chemicon International, Temecula, Calif.) as a positive control DNA.

### Cloning and Expression of Human MGMT

The human MGMT cDNA (pSV2MGMT) was amplified by PCR using primers hMGMT-F (CACCATGGACAAGGATTGTGAAAT; SEQ ID NO: 7) and hMGMT-R (CTAGTTTCGGCCAGCAGGCG; SEQ ID NO: 8). MGMT cDNA was then cloned via a Topoisomerase cloning procedure into the pENTR-D cloning plasmid (Invitrogen, Carlsbad, Calif.), as per the manufacturers protocol. The human MGMT open reading frame (ORF) was transferred from pENTR-hMGMT to a Gateway modified pIRES-Puro plasmid via LR recombination reaction, as per the manufacturer (Invitrogen, Carlsbad, Calif.).

Expression of human MGMT: briefly, 1.5×10⁵ cells were seeded into 60 mm dishes and incubated for 24-30 hours at 5% CO₂ at 37° C. The human MGMT expression plasmid (pIRES-Puro-hMGMT) was transfected using FuGene™ 6 Transfection Reagent (Roche, Indianapolis, Ind.) according to the manufacturer's instructions. Stable cell lines were selected in puromycin (0.5 μg/ml) for 2 weeks, individual clones (stably expressing human MGMT) were amplified and 30 μg of nuclear extract was analyzed by immunoblot analysis for the expression of human MGMT protein.

### Example 2

### MPG Over-Expression Enhances BER Inhibition-Mediated Sensitization of Glioma Cells to TMZ

### MX-Induced Potentiation of TMZ was Enhanced by Over-Expression of MPG in Glioma Cells

To test the hypothesis that increased repair initiation by MPG will further sensitize glioma cells exposed to BER inhibition, WT MPG was stably over-expressed in the glioma cell line LN428. Over-expression of MPG was confirmed at the protein and mRNA levels using both immunoblotting (FIG. 1A) and qRT-PCR analyses (FIG. 1B), with an approximate 40-fold increase of both protein (as determined by measuring immunoblot band intensity using Image J software) and mRNA. To confirm increased glycosylase activity in the MPG over-expressing LN428/MPG cells, a fluorescent MPG activity assay was developed using a modified form of molecular beacons, similar to that previously reported for oxidative damage (Maksimenko et al., Biochem. Biophys. Res. Commun., 319:240-246, 2004). These molecular beacon repair substrates are stem-loop structures formed by single-stranded DNA with a fluorophore (6-FAM) and a quencher (Dabcyl) on either end of the DNA. A 3-ethoadenine lesion (8A), a substrate of MPG, was positioned in the stem region of the beacon at base #5 from the 5′ end and was used to probe for MPG activity. The same beacon structure with a normal adenine was used as the control substrate. MPG activity was assayed by measuring fluorescence signal which was produced as a result of breaking apart the fluorophore from the quencher, due to removal of the 3-etho-adenine by MPG and subsequent DNA strand excision by APE1 (FIG. 1C). Results of these experiments demonstrated that the LN428/MPG cell line (FIG. 1D, filled circle) had significantly higher MPG activity as compared to the LN428 cell line (FIG. 1D, filled rectangle), while cell extracts from both cell lines showed no activity over the control substrate (FIG. 1D, empty symbols).

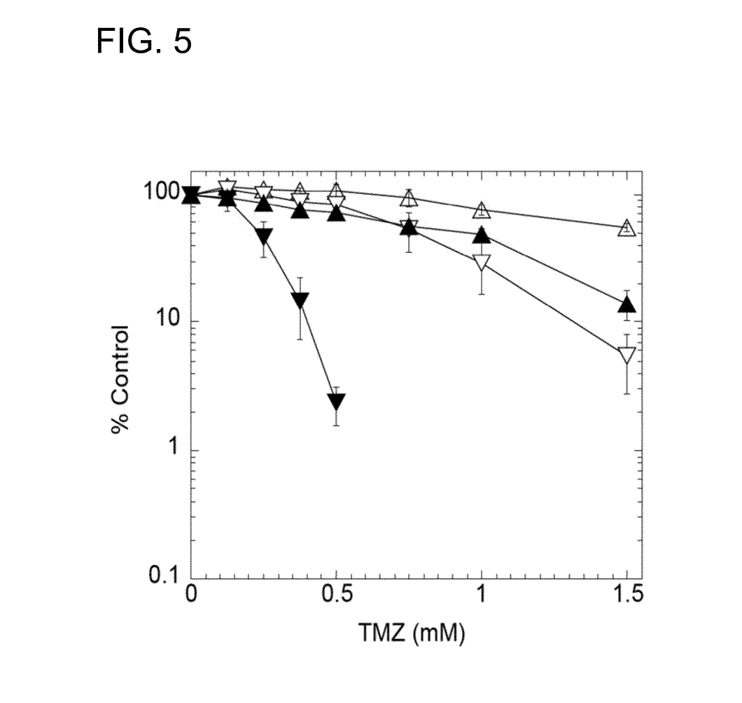

Using a short-term cell survival assay (48 hours MTS assay), the potentiation of TMZ by MX was assayed in the LN428 cells with or without MPG over-expression. MX sensitized both cell lines to TMZ. However, the potentiation of TMZ induced by MX was much higher in the MPG over-expressing cells as compared to the non-over expressing cells (FIG. 1E and FIG. 5). To confirm that MPG over-expression-induced potentiation is a result of elevated glycosylase activity, a mutant MPG (N169D), which has almost no glycosylase acclivity (Connor et al., Chem. Res. Toxicol., 18:87-94, 2005), was over-expressed in the glioma cell line LN428 (Tang et al., Mol. Cancer Res. 8(1):67-79, 2010). Overexpression of the mutant MPG did not sensitize LN428 cells to a combined treatment of MX and TMZ (FIG. 1F), which supports the hypothesis that MPG over-expression induced sensitization is due to increased glycosylase activity in the cells.

### MX-Induced Potentiation of TMZ is Regulated by the Expression of Polβ

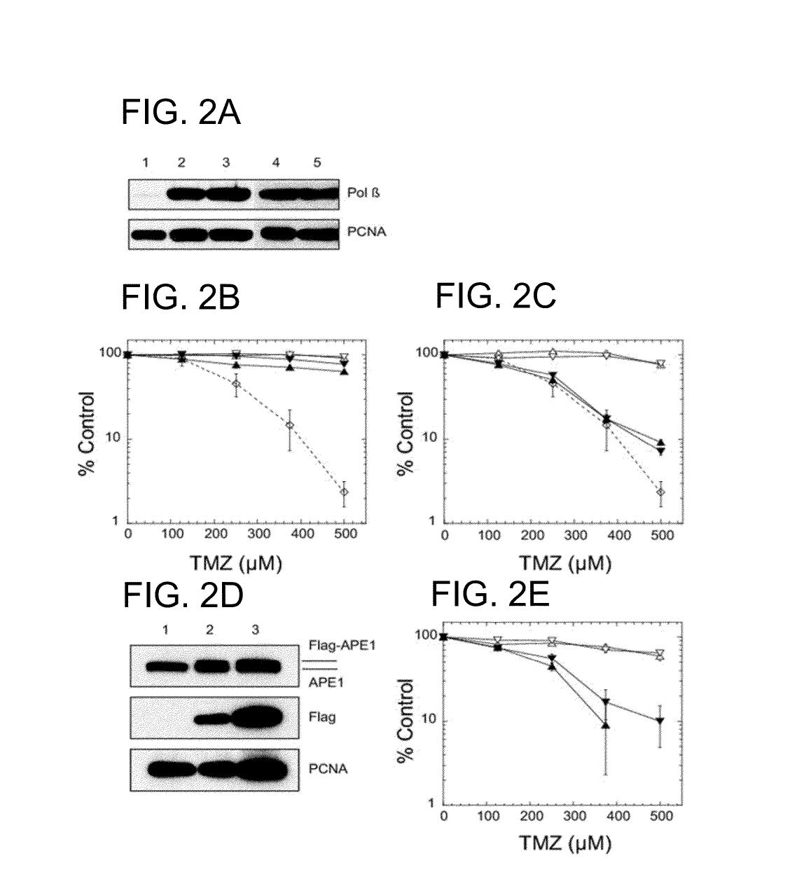

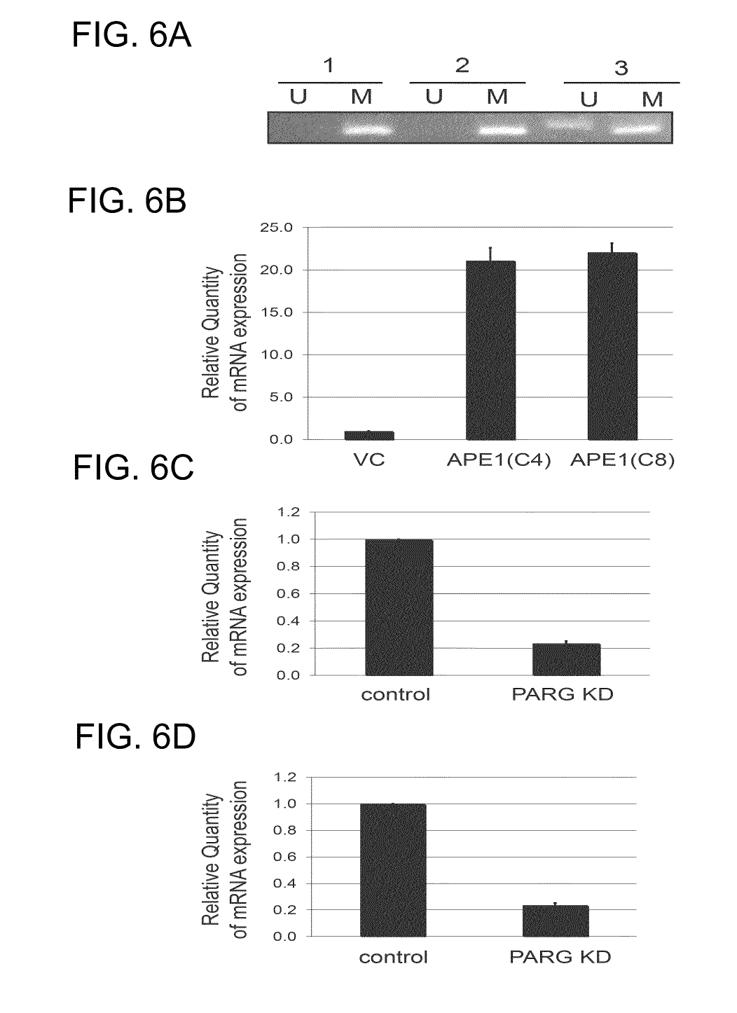

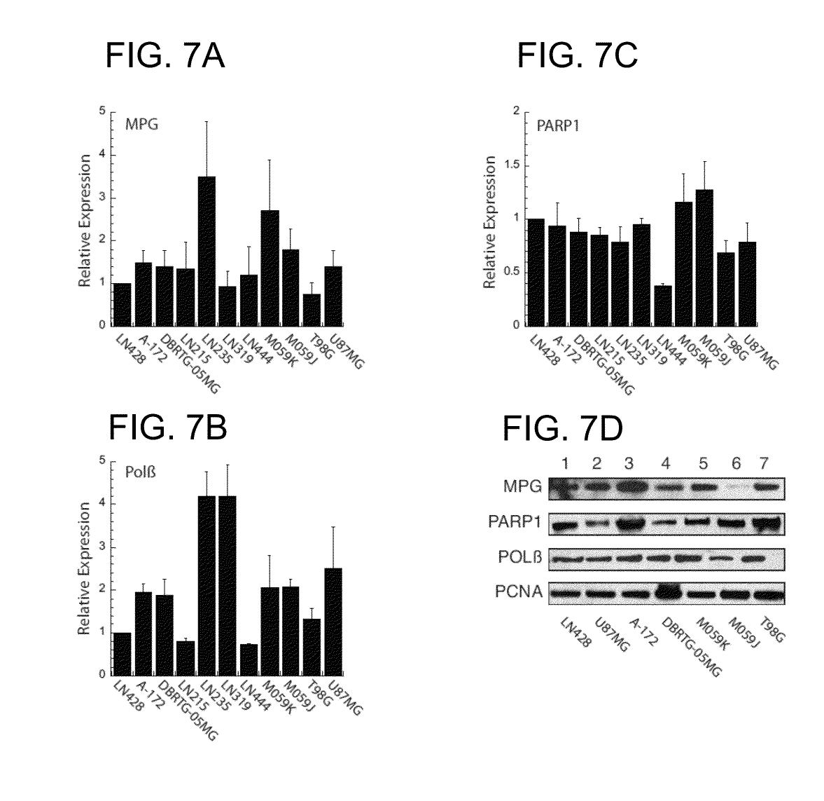

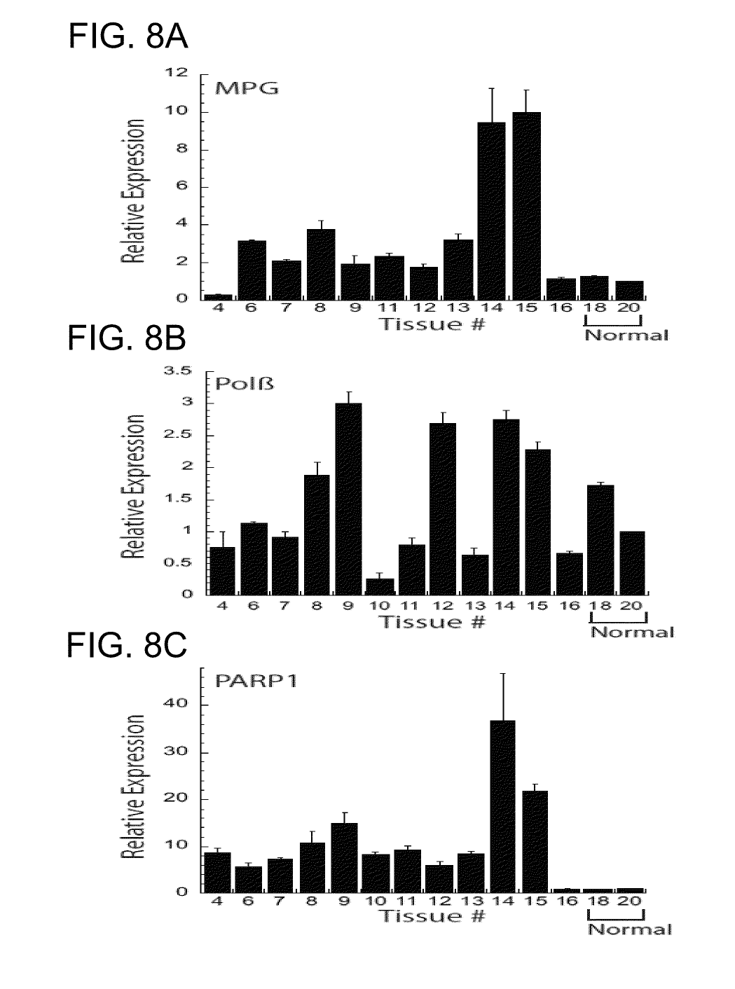

Although MX can efficiently react with AP sites in vitro (Rosa et al., Nucleic Acids Res., 19:5569-5574, 1991), it doesn't exclude the possibility that a proportion of the AP sites produced in cells following TMZ exposure will be processed by APE1 and the subsequent steps of BER. To investigate the impact of processing of AP sites by BER proteins on MX-induced potentiation of TMZ, Polβ, the rate-limiting enzyme of the BER pathway (Sobol et al., J. Biol. Chem., 278:39951-39959, 2003), was re-expressed and MX-induced potentiation in these cell lines was assessed. Over-expression of WT Polβ in the LN428/MPG cells (FIG. 2A) dramatically reduced the potentiation induced by MX (FIG. 2B, compare to FIG. 1E). In contrast, over-expression of a 5′ dRP lyase null mutant (K72A) (Sobol et al., Nature, 405:807-810, 2000; Sobol et al., Nature, 379:183-186, 1996) of Polβ (FIG. 2B) did not affect MX-induced potentiation of TMZ (FIG. 2C). Further, to determine whether increased expression of APE1 affects MX-induced potentiation of TMZ, APE1 was over-expressed in the LN428/MPG cells (FIG. 2D and FIG. 6B).

Interestingly, increased expression of APE1 did not alter the potentiation of TMZ induced by MX (FIG. 2E). A possible explanation for the observation is that although over-expression of APE1 increased its mRNA level by 20-fold, its protein level has only been slightly increased, which might not be able to significantly increase the number of AP sites react with APE1 instead of MX (FIG. 2D and FIG. 6B).

### PARG Deficiency-Induced Potentiation of TMZ is Enhanced by Over-Expression of MPG in the Presence of MGMT

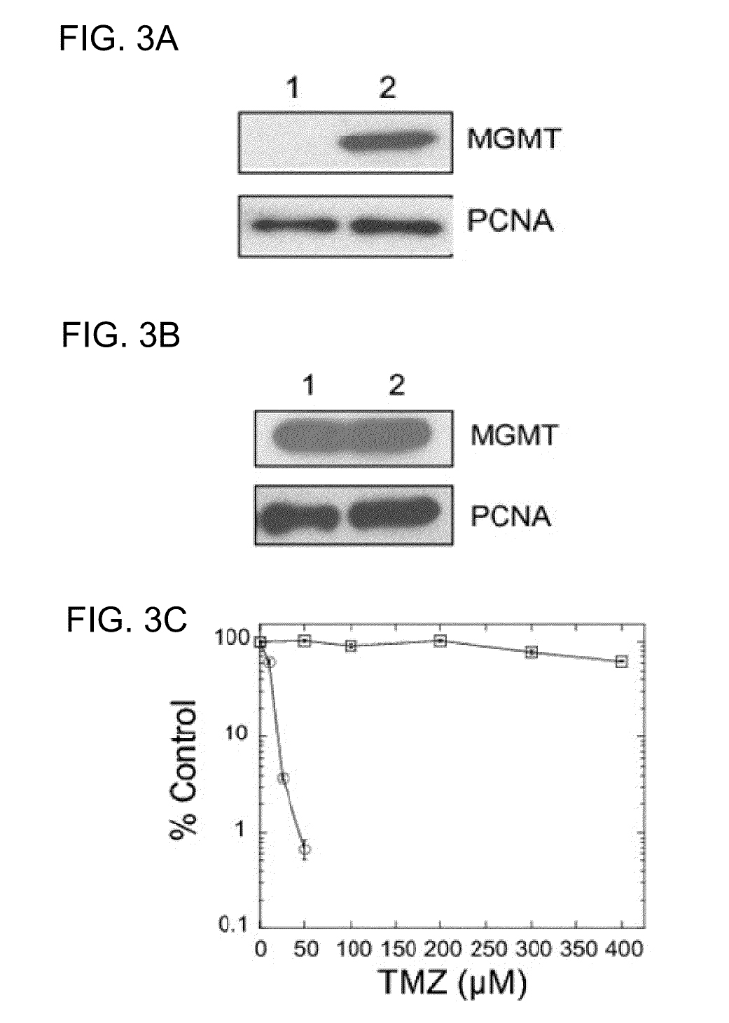

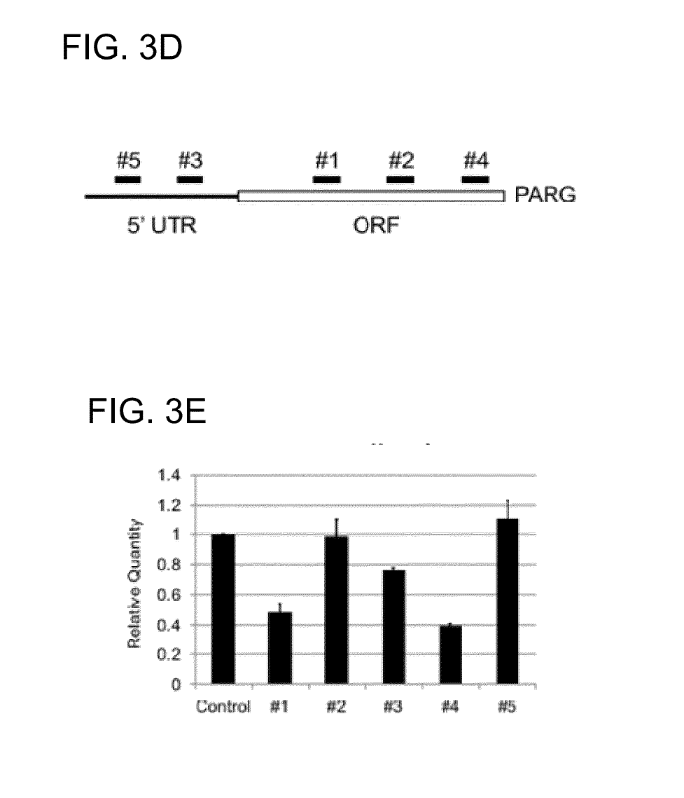

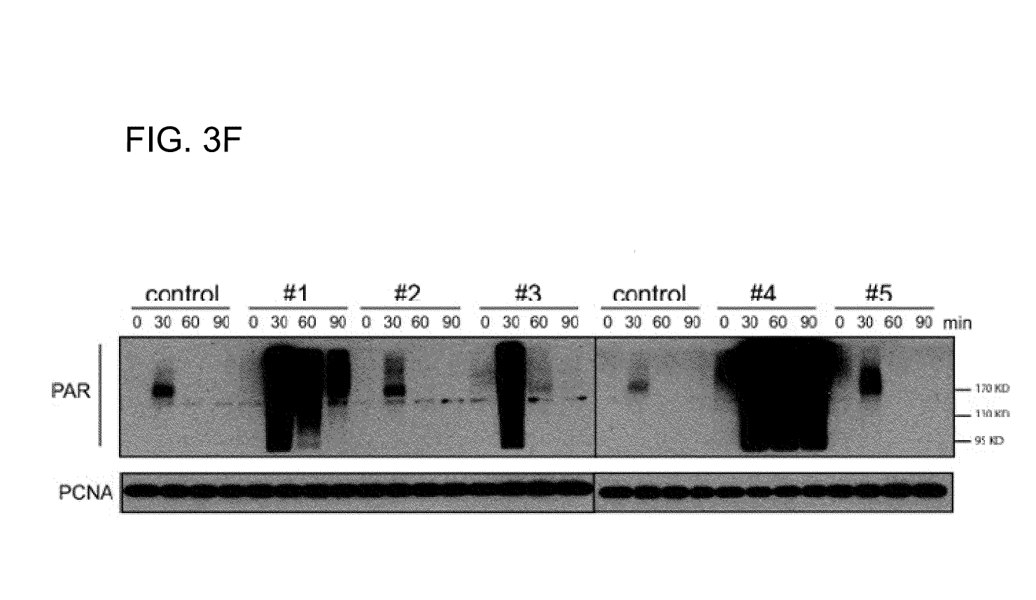

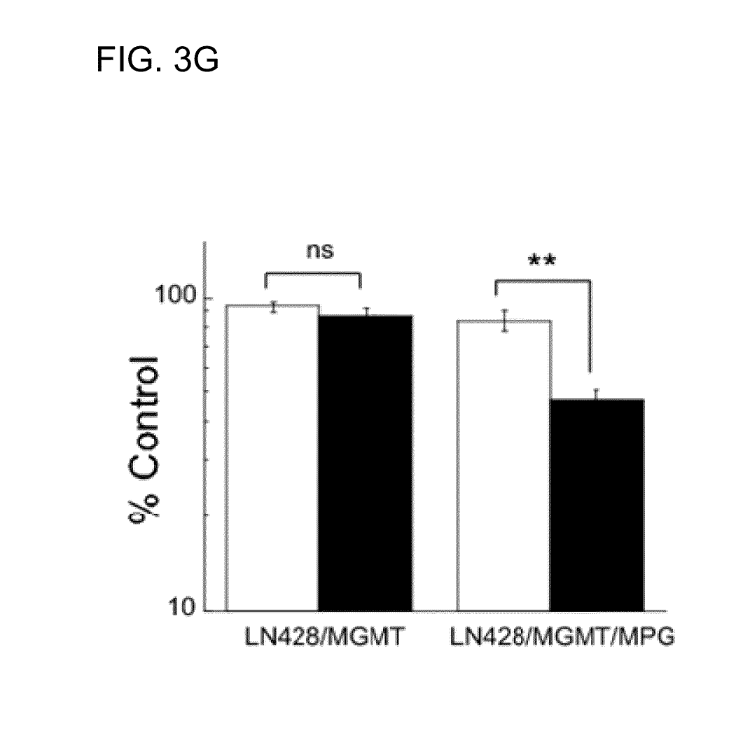

This study addresses chemotherapy sensitization in a MGMT background for targeting the other 50% gliomas that express MGMT and those with mismatch repair (MMR) deficiency regardless of the MGMT status. The LN428 cell line used in the study has no detectable expression of MGMT (FIG. 3A) as a result of epigenetic silencing by promoter methylation (FIG. 6A). To study BER inhibition-induced chemotherapy potentiation in the presence of MGMT expression, the LN428 and LN428/MPG cells were transfected with a mammalian expression plasmid (pIRES-Puro-hMGMT) and a cell clone stably expressing MGMT was selected for further experiments (FIG. 3B). Over-expression of MGMT provided LN428 cells resistance to TMZ as shown in FIG. 3C.

Although poly(ADP-ribosyl)ation of PARP1 and other BER proteins, for instance XRCC1, facilitates the repair of base lesions, the dynamics between PAR synthesis and degradation is also important for the effectiveness of the repair process (Tentori et al., Eur. J. Cancer, 41:2948-2957, 2005). Previously, it has been reported that a deficiency in the degradation of PAR negatively affects the repair of base lesions and sensitized cells to base damage (Fisher et al., Mol. Cell Biol., 27:5597-5605, 2007). In this study, it was investigated whether deficiency in PAR degradation-induced potentiation of TMZ can be further increased by over-expression of MPG or not. Because PARG is the primary enzyme responsible for degrading PAR in vivo, five different shRNA constructs targeting PARG (FIG. 3D) were first screened using an HIV-lentiviral system (Zufferey et la., J. Virol., 72:9873-9880, 1998; Zufferey et al., Nat. Biotechnol., 15:871-875, 1997) in the LN428/MPG cells for effective KD of the enzyme.

Using RNA samples prepared from LN428/MPG cells stably expressing each of the five shRNA, qRT-PCR results showed that the cells expressing #4 and #2 constructs have the lowest and the second lowest level of PARG mRNA (FIG. 3E). To assay the impact of PARG shRNA expression on the ability of cells to degrade DNA damage-induced PAR formation, control cells and cells treated with 1.5 mM TMZ were lysed at different time points and the lysates were probed for PAR in immunoblot analyses. Consistent with the qRT-PCR results, PARG shRNA #2 and #4 greatly decreased the degradation of PAR following exposure to TMZ (FIG. 3F). Based on these results, the #4 construct for effective PARG KD was used in the following experiments. Further, to minimize the O⁶-methylguanine-induced cytotoxicity and to target tumor cells with expression of MGMT or with MMR deficiency for sensitization, PARG KD-induced potentiation of TMZ in the LN428 cell lines with re-expressed MGMT was assessed. First, stable PARG KD was generated in the MGMT expressing LN428 and LN428/MPG cell lines as determined by qRT-PCR using the #4 PARG shRNA lentivirus construct (FIGS. 6C and 6D). Next, using long-term cell survival assays, PARG KD-induced potentiation of TMZ was assayed in these cell lines. The results demonstrated that a deficiency in degrading PAR as a result of PARG KD significantly sensitized cells to TMZ in the MPG over-expressing cells (LN428/MGMT/MPG), while sensitization by PARG KD was not observed in the parental cells with a low level of MPG expression (LN428/MGMT) (FIG. 3G).

### PARP Inhibitor-Induced Potentiation of TMZ is Enhanced by Over-Expression of MPG

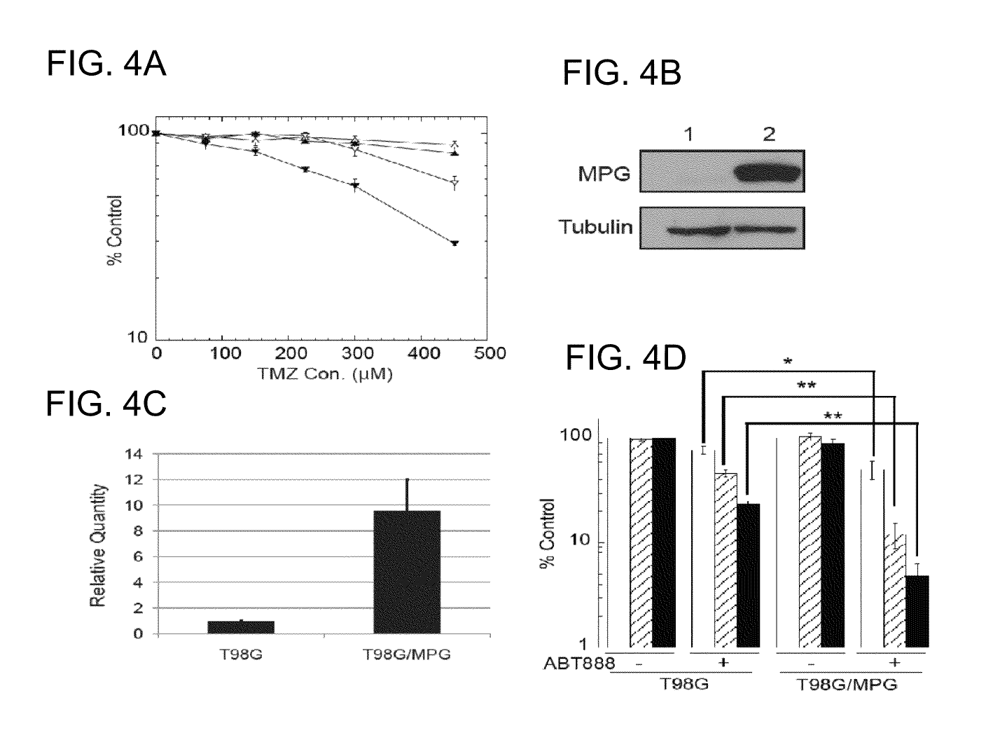

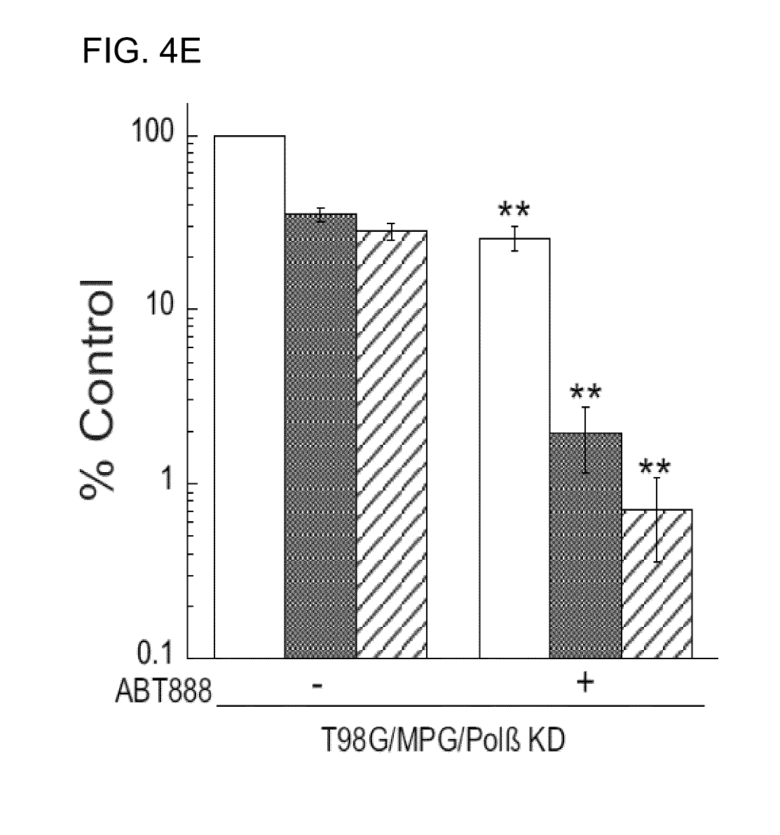

Using a long-term cell survival assay, whether PARP inhibitor-induced potentiation of TMZ is affected by over-expression of MPG was assessed. It has been previously shown that PARP inhibitor PJ34 at 2 μM concentration significantly reduced the level of PARP activation following exposure to TMZ (Tang et al., Mol. Cancer Res. 8(1):67-79, 2010). Here it is shown that pre- (4 μM) and co-treatment with PJ34 (2 μM) sensitized cells to TMZ, and this sensitization was much higher in the MPG over expressing cells (LN428/MGMT/MPG) as compared to the parental cells with low level of MPG expression (LN428/MGMT) (FIG. 4A). To further confirm that over-expression of MPG increases PARP inhibition-induced potentiation of TMZ in glioma cells, another glioma cell line T98G was used, which cell line has endogenous expression of MGMT (FIGS. 3A & B), and a clinical relevant PARP inhibitor ABT-888 for similar experiments as those been conducted in the LN428 cell lines with PJ34. MPG was over-expressed in the T98G cells using a mammalian expression plasmid (pRS1422). Over-expression of MPG in the T98G cells increased its mRNA level (10-fold) and protein level as determined by immunoblotting and qRT-PCR analysis (FIG. 4B & C). Consistent with previous reports that demonstrate ABT-888 potentiates TMZ in diverse tumor models (Donawho et al., Clin. Cancer Res., 13:2728-2737, 2007; Palma et al., Clin. Cancer Res., 2009), treatment with ABT-888 sensitized T98G cells to TMZ (FIG. 4D). More importantly, over-expression of MPG significantly increased the potentiation induced by ABT-888 (FIG. 4D, p<0.05 and p<0.01).

Depletion of Polβ in the MPG over-expressing T98G cells (T98G/MPG/Polβ KD) enhanced the ABT-888 mediated sensitization of the cells to TMZ treatment (IC₅₀<25 μM). Similar to the T98G/MPG cells, ABT-888 treatment alone resulted in cell killing in the T98G/MPG/Polβ KD cells, albeit the killing effect was much stronger as it killed about 70% cells as compared to 30% in the T98G/MPG cells (FIG. 4E and FIG. 4B). A combined treatment with TMZ and ABT-888 in the T98G/MPG/Polβ KD cells induced significantly increased cytotoxicity compared to TMZ treatment alone (FIG. 4E, p<0.01), suggesting that the expression status of Polβ also plays a role in determining ABT-888-induced potentiation of TMZ. These results demonstrate that increased BER repair initiation enhances PARP inhibitor-induced potentiation of TMZ via a process that is also dependent on the expression of Polβ. Hence, the expression level of both MPG and Polβ in tumors can be used to as a biomarker for alkylator chemotherapy potentiation by methoxyamine or PARP inhibitors.

These functional and drug-induced cytotoxicity analyses prompted the inventors to next determine if glioma cell lines and glioma tumors present with varying levels of expression for MPG, Polβ and PARP1 mRNA and/or protein. Additional established glioma cell lines were obtained and the mRNA expression of MPG, Polβ and PARP1 by analyzed qRT-PCR. As shown (FIGS. 5A-5C), the mRNA expression was variable across the eleven cell lines. Both MPG and Polβ mRNA expression varied as much as 4-fold, as compared to the LN428 cell line, whereas PARP1 mRNA expression was relatively constant. In some cases, it was possible to also analyze protein expression by immunoblot. As shown in FIG. 5D, Polβ protein expression was relatively constant whereas variations in protein expression was observed for MPG and PARP1. It should be noted that the relationship between mRNA and protein expression is not always 1:1, as suggested previously (Gygi et al., Mol Cell Biol 19:1720-1730, 1999). The mRNA expression pattern in the GBM tumors was considerably more varied. In this analysis, expression was normalized to the expression of each mRNA in a normal brain tissue sample (FIG. 6). Both normal brain samples presented with relatively similar expression levels for all three mRNAs analyzed. However, the tumor tissue showed significant variability in expression of these key BER genes: MPG mRNA expression varied as much as 10-fold (FIG. 6A), Polβ mRNA expression varied as much as 8-fold (FIG. 6B) and PARP1 mRNA expression varied as much as 40-fold, as compared to normal brain (FIG. 6C).

### Summary of Results

As the very first enzyme in the BER pathway, MPG activity is required for the initiation of BER to repair a spectrum of alkylated bases (Wood et al., Science, 291:1284-1289, 2001). It has been demonstrated that MPG expression levels vary considerably in human breast cancer (Cerda et al., FEBS Lett., 431:12-18, 1998), in astrocytic tumors (Kim et al., Anticancer Res., 23:1417-1423, 2003) and in glioblastomas. In addition, MPG possesses multiple post-translational modifications and interactions with many DNA repair proteins, including XRCC1 and hR23A, suggesting that the glycosylase activity of MPG available for the BER process may be under cellular regulation (Almeida and Sobol, DNA Repair (Amst), 6:695-711, 2007).

Herein it is demonstrated that BER inhibition-mediated sensitization of glioma cells to chemotherapeutic agent TMZ was further increased by increasing BER repair initiation via over-expression of MPG. Glioma cells with overexpression of MPG exhibited dramatically increased potentiation of TMZ via BER inhibition by MX, the PARP inhibitors PJ34 and ABT-888, or by PARG depletion (PARG KD). The enhanced potentiation of TMZ in the MPG over-expressing glioma cell line observed in these studies is in line with a previous report showing that MX-induced sensitization is increased by MPG over-expression in ovarian cancer cells (Fishel et al., Clin. Cancer Res., 13:260-267, 2007). However, expression level of MPG may not be the only factor that controls MX-induced potentiation of TMZ, as it might also be related to the efficiency of the BER pathway that process AP site and its downstream repair intermediates. From the experiments disclosed herein (FIGS. 2C & D), it was shown that re-expression of the wild type BER rate-limiting enzyme Polβ, but not the 5′ dRP lyase activity null mutant (K72A), in the MPG over-expressing cells abrogated the MPG dependent potentiation. Therefore, it is the collective expression status of both MPG and Polβ that defines the sensitization induced by MX. APE1 is the main enzyme that directly competes with MX for the processing of AP sites in cells; over-expression of the enzyme did not alter MX-induced potentiation of TMZ (FIG. 2D). A possible explanation might be that although APE1 mRNA level was increased by more than 20× (FIG. 6B), the protein level of APE1 has only been slightly increased (FIG. 2E). Since APE1 is an abundant enzyme in cells, a slight increase of the protein level of APE1 may not change the ratio of AP sites processed by APE1 or MX.

According to a previous study of the inventors (Tang et al., Mol. Cancer Res. 8(1):67-79, 2010), the dynamics between PAR synthesis and degradation is not only involved in facilitating repair of base lesions, but also acts as a mediator of cell death via over synthesis of PAR and subsequent cellular energy depletion in response to accumulation of un-repaired BER intermediates. Thus, although inhibition of over-activation of PARP and PAR synthesis provides short-term cell survival advantage, it is believed that damage-induced DNA lesions persist in cells due to inhibition of the role of PARP in repair. Cells harboring the un-repaired DNA lesions eventually will die due to DSBs accumulation as cells go through replication. Therefore, in the context of chemotherapy sensitization involving PARP inhibition or depletion of PARG (PARG KD), the long-term assay (10 days) for cell survival, which allows for replication of cells, is more suitable than the short-term (48 hours) MTS assay. For the reasons stated above, all the cell survival assays involving PARG or PARP inhibition were conducted using the long-term assay as described in Example 1.

PARG is the primary enzyme for degrading PAR synthesized in cells. It has been reported that PARG inhibitor serves as a chemosensitizer of malignant melanoma for TMZ (Tentori et al., Eur. J. Cancer, 41:2948-2957, 2005), which implies that not only poly(ADP-ribosyl)ation of target proteins by PARP but also the clearance of the modification by PARG in a timely manner is important for cell survival following DNA base damage. In line with the previous report which demonstrates PARG inhibition sensitizes melanoma to TMZ (Tentori et al., Eur. J. Cancer, 41:2948-2957, 2005), it is disclosed herein that shRNA-mediated PARG down regulation sensitizes glioma cells to TMZ. More importantly, it is shown that the sensitization is greatly enhanced in cells with overexpression of MPG (FIG. 3G).

PARP has recently become the focus of investigations of chemotherapy potentiation since the publication of an extremely sensitive phenotype induced by PARP inhibitors in breast cancer cells bearing a loss of BRCA1 or BRCA2 function (Bryant et al., Nature, 434:913-917, 2005; Farmer et al., Nature, 434:917-921, 2005). Currently, PARP inhibitors are under phase 0 to phase 2 clinical trials in combination with clinical alkylating agent TMZ (Ratnam & Low, Clin. Cancer Res., 13:1383-1388, 2007). The rationale for combining PARP inhibitor with TMZ is generally considered to be inhibition of repair of TMZ-induced DNA lesions via inhibiting PARP. However, it is not known if the status of the BER pathway inherent in cancer cells has an impact on the potentiation induced by PARP inhibitors. In this study, using PARP inhibitors PJ34 and ABT888, it is demonstrated that PARP inhibition-induced potentiation of TMZ is greatly increased in glioma cells with over-expression of MPG (FIG. 4A), suggesting that increased initiation of repair of TMZ-induced base lesions can further sensitize cancer cells to PARP inhibition and the expression level of MPG in cancer cells may foretell the extent of sensitization PARP inhibitors could achieve.

The experiments disclosed herein address the relationship between the repair initiation of BER and chemotherapy sensitization via BER inhibition, by using MX, the PARP inhibitor PJ34 and ABT888, or by PARG KD. It is demonstrated that the BER inhibition-induced potentiation of TMZ is enhanced by over-expression of the BER initiating enzyme MPG, suggesting that combining an increase in repair initiation and inhibition of repair following initiation of the BER pathway might be an effective means to improved chemotherapy efficacy and the expression level of MPG in cancer cells might be used to predict the effectiveness of a treatment that combines BER inhibition with clinically used alkylating agents.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A method of determining if a subject with cancer will be sensitive to temozolomide (TMZ) and methoxyamine, or TMZ and a poly(ADP-ribose) polymerase (PARP) inhibitor, comprising:
measuring expression of polymerase β (Polβ) and N-methylpurine DNA glycosylase (MPG) in a sample obtained from the subject;
detecting a decrease in expression of Polβ and an increase in expression of MPG relative to a control; and
correlating a decrease in expression of Polβ and an increase in expression of MPG relative to the control with sensitivity of the subject to TMZ and methoxyamine, or to TMZ and the PARP inhibitor, thereby determining that the subject is sensitive to TMZ and methoxyamine, or to TMZ and the PARP inhibitor.

2. The method of claim 1, comprising determining if the subject will be sensitive to TMZ and methoxyamine.

3. The method of claim 1, comprising determining if the subject will be sensitive to TMZ and a PARP inhibitor.

4. The method of claim 1, wherein the cancer is a solid tumor or a hematologic cancer.

5. The method of claim 4, wherein the solid tumor is a central nervous system (CNS) tumor, a lymphoma, melanoma, osteosarcoma, colorectal cancer, lung cancer, ovarian cancer, breast cancer or HPV-infected neoplasia.

6. The method of claim 5, wherein the CNS tumor is a glioma, medulloblastoma, astrocytoma, germinoma, meningioma, oligodendroglioma, Schwannoma, craniopharyngioma, ependymoma or CNS lymphoma.

7. The method of claim 4, wherein the hematologic cancer is a leukemia.

8. The method of claim 1, wherein the PARP inhibitor is a small molecule inhibitor, antisense molecule or antibody.

9. The method of claim 8, wherein the antisense molecule is an antisense oligonucleotide, siRNA, miRNA or ribozyme specific for PARP.

10. The method of claim 8, wherein the small molecule PARP inhibitor is PJ34, ABT-888, AG14699, AG14361, CEP-6800, CEP-8983, INO-1001, KU59436, BSI-201, GPI 21016, GPI15427 or AZD2281.

11. The method of claim 1, wherein the sample is a tumor biopsy.

12. The method of claim 1, wherein detecting a decrease in expression of Polβ and an increase in expression of MPG comprises measuring the level of Polβ and MPG mRNA.

13. The method of claim 1, wherein detecting a decrease in expression of Polβ and an increase in expression of MPG comprises measuring the level of Polβ and MPG protein.

14. The method of claim 1, wherein detecting a decrease in expression of Polβ and an increase in expression of MPG comprises measuring functional activity of Polβ and MPG.

15. The method of claim 1, wherein the control is a control sample obtained from a subject that is not sensitive to TMZ and methoxyamine, or TMZ and a PARP inhibitor.

16. The method of claim 1, wherein the control is a reference value.

17. The method of claim 1, wherein the subject is determined to be sensitive to TMZ and methoxyamine, or TMZ and a PARP inhibitor, and the method further comprises administering TMZ in combination with methoxyamine, or TMZ in combination with the PARP inhibitor, to the subject.

18. The method of claim 1, wherein detecting a decrease in expression of Polβ and an increase in expression of MPG comprises measuring the level of Polβ and MPG mRNA by RT-PCR, or comprises measuring the level of Polβ and MPG protein by immunoblot.

19. The method of claim 1, wherein detecting a decrease in expression of Polβ and an increase in expression of MPG comprises isolating RNA from the sample obtained from the subject and measuring the level of Polβ and MPG mRNA by RT-PCR.

20. The method of claim 1, wherein detecting an increase in expression of MPG comprises measuring glycosylase activity of MPG using an oligonucleotide comprising the nucleotide sequence of SEQ ID NO: 2, wherein the oligonucleotide further comprises carboxyfluorescein at the 5′ end and 4-(4′ dimethylaminophenylazo) benzoic acid at the 3′ end.


SEQUENCE LISTING (13 sequences)

SEQ ID NO: 1 - DNA, 43 bp, Artificial Sequence
  Synthetic oligonucleotide
gcactattgaattgacacgccatgtcgatcaattcaatagtgc

SEQ ID NO: 2 - DNA, 43 bp, Artificial Sequence
  Synthetic oligonucleotide
gcactnttgaattgacacgccatgtcgatcaattcaatagtgc

SEQ ID NO: 3 - DNA, 23 bp, Artificial Sequence
  Synthetic oligonucleotide
tttcgacgttcgtaggttttcgc

SEQ ID NO: 4 - DNA, 22 bp, Artificial Sequence
  Synthetic oligonucleotide
gcactcttccgaaaacgaaacg

SEQ ID NO: 5 - DNA, 29 bp, Artificial Sequence
  Synthetic oligonucleotide
tttgtgttttgatgtttgtaggtttttgt

SEQ ID NO: 6 - DNA, 28 bp, Artificial Sequence
  Synthetic oligonucleotide
aactccacactcttccaaaaacaaaaca

SEQ ID NO: 7 - DNA, 24 bp, Artificial Sequence
  Synthetic oligonucleotide
caccatggacaaggattgtgaaat

SEQ ID NO: 8 - DNA, 20 bp, Artificial Sequence
  Synthetic oligonucleotide
ctagtttcggccagcaggcg

SEQ ID NO: 9 - DNA, 58 bp, Artificial Sequence
  Synthetic oligonucleotide
ccgggctaagatgaaatcggagtatctcgagatactccgatttcatcttagctttttg

SEQ ID NO: 10 - DNA, 58 bp, Artificial Sequence
  Synthetic oligonucleotide
ccggcgattgcatgtcacttacgaactcgagttcgtaagtgacatgcaatcgtttttg

SEQ ID NO: 11 - DNA, 58 bp, Artificial Sequence
  Synthetic oligonucleotide
ccgggcctaggaaattctcctccatctcgagatggaggagaatttcctaggctttttg

SEQ ID NO: 12 - DNA, 58 bp, Artificial Sequence
  Synthetic oligonucleotide
ccgggctgagcgagatgtggtttatctcgagataaaccacatctcgctcagctttttg

SEQ ID NO: 13 - DNA, 58 bp, Artificial Sequence
  Synthetic oligonucleotide
ccgggcagtttagtaatgctaacatctcgagatgttagcattactaaactgctttttg